(19) United States
(12) Patent Application Publication
(10) Pub. No.: US 2026/0159505 A1
(43) Pub. Date: Jun. 11, 2026

(54) ROCK2 INHIBITORS AND USES THEREOF

(71) Applicant: Restor Pharmaceuticals, Falls Church, VA (US)

(72) Inventors: Enoch KIM, Marblehead, MA (US); Paul SWEETNAM, Marblehead, MA (US); Bridget McCarthy Cole, Quincy, MA (US)

(21) Appl. No.: 19/316,900

(22) Filed: Sep. 2, 2025

Related U.S. Application Data
(60) Provisional application No. 63/690,063, filed on Sep. 3, 2024.

(51) Int. Cl. C07D 403/12; A61K 31/517; A61K 31/5377; C07B 59/00; C07D 401/14; C07D 413/14

(52) U.S. Cl. CPC C07D 403/12; A61K 31/517; A61K 31/5377; C07B 59/002; C07D 401/14; C07D 413/14

(57) ABSTRACT

The present invention relates to compounds having the Formula

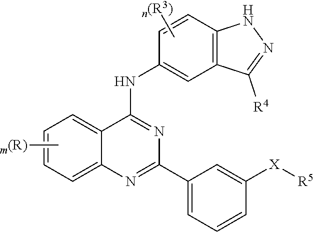

wherein the variables are defined herein. Pharmaceutically acceptable salts, compositions, methods of use and uses to make medicaments thereof.

### RELATED APPLICATION

This application claims the benefit of U.S. Provisional Application No. 63/690,063, filed on Sep. 3, 2024. The entire teachings of the above application are incorporated herein by reference.

### BACKGROUND OF THE INVENTION

Rho-associated kinase is an intracellular regulator of cytoskeletal dynamics and cell motility. Rho-kinase regulates a number of downstream targets of RhoA through phosphorylation, including, for example, myosin light chain, the myosin light chain phosphatase binding subunit and LIM-kinase 2. In smooth muscle cells Rho-kinase mediates calcium sensitization and smooth muscle contraction. Inhibition of Rho-kinase blocks 5-HT and phenylephrine agonist induced muscle contraction. When introduced into non-smooth muscle cells, Rho-kinase induces stress fiber formation and is required for the cellular transformation mediated by RhoA. Rho-kinase participates in a variety of cellular processes, including but not limited to Na/H exchange transport system activation, stress fiber formation, and adducing activation. Rho-kinase is involved in physiological processes such as vasoconstriction, bronchial smooth muscle constriction, vascular smooth muscle and endothelial cell proliferation, platelet aggregation, and others.

Inhibition of Rho-kinase activity in animal models has demonstrated a number of benefits of Rho-kinase inhibitors for the treatment of human diseases. These include models of cardiovascular diseases such as obesity, metabolic disorder, energy homeostasis, immunological disorders, hypertension, atherosclerosis, restenosis, cardiac hypertrophy, ocular hypertension, cerebral ischemia, cerebral vasospasm, penile erectile dysfunction, central nervous system disorders such as neuronal degeneration and spinal cord injury, and in neoplasias where inhibition of Rho-kinase activity has been shown to inhibit tumor cell growth and metastasis, angiogenesis, arterial thrombotic disorders such as platelet aggregation and leukocyte aggregation, asthma, regulation of intraocular pressure, and bone resorption. The inhibition of Rho-kinase activity in patients has benefits for controlling cerebral vasospasms and ischemia following subarachnoid hemorrhage. Belumosudil is an FDA-approved Rho-kinase inhibitor and is described in U.S. Pat. No. 8,357,693, which is incorporated herein by reference.

Casein kinase II (CK2) is a kinase with a brain-enriched variant that shows increased activity in inflammatory and neurodegenerative diseases. CK2 has been shown to regulate mitochondrial homeostasis as well as innate immune pathways. CK2 can regulate innate immunity pathways, and CK2 inhibitors have shown efficacy in immune-driven cancers. CK2 protein levels increase in peripheral immune cells upon induction by pro-inflammatory stimuli such as LPS and TNF alpha, and CK2 phosphorylates important mediators of inflammation, including NF-KB, IKBa and AKT.

Inhibition of CK2 activity has demonstrated a number of benefits for the treatment of human diseases, including inflammation, autoimmune disorders, diabetes, obesity and metabolic disorder and fibrosis.

### SUMMARY OF THE INVENTION

The present invention relates to compounds having the Formula (I):

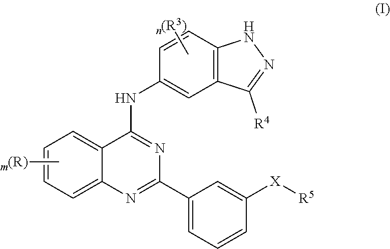

or a pharmaceutically acceptable salt thereof, wherein:
each R is independently H, deuterium, halogen, alkyl (such as C₁₋₆ alkyl), haloalkyl (such as C₁₋₆ haloalkyl), carbocyclyl (such as C₃₋₇ carbocyclyl), heterocyclyl (such as 3-7 membered heterocyclyl), nitrile or —OR⁷, wherein each alkyl, haloalkyl, carbocyclyl, or heterocyclyl is independently optionally substituted, wherein at least one R is selected from deuterium, halogen, alkoxy, trifluoromethyloxy or nitrile;
each R³ is independently deuterium, halogen, alkyl (such as C₁₋₆ alkyl), haloalkyl (such as C₁₋₆ haloalkyl), carbocyclyl (such as C₃₋₇ carbocyclyl), hetercyclyl (such as 3-7 membered heterocyclyl), or —OR⁷, wherein each alkyl, haloalkyl, carbocyclyl, or heterocyclyl is independently optionally substituted;
n is 0, 1, 2, or 3;
m is 0, 1, 2, 3 or 4;
X is —CON(R⁶)— or —N(R⁶)CO—
R⁵ and R⁶ are each independently H, alkyl (such as C₁₋₆ alkyl), haloalkyl (such as C₁₋₆ haloalkyl), carbocyclyl (such as C₃₋₇ carbocyclyl), or heterocyclyl (such as 3-7 membered heterocyclyl), or R⁵ and R⁶ are joined together to form 3-7 membered heterocyclyl, wherein each alkyl, haloalkyl, carbocyclyl, or heterocyclyl is independently optionally substituted; and
each R⁷ is independently H, alkyl (such as C₁₋₆ alkyl), haloalkyl (such as C₁₋₆ haloalkyl), carbocyclyl (such as C₃₋₇ carbocyclyl), or heterocyclyl (such as 3-7 membered heterocyclyl), wherein each alkyl, haloalkyl, carbocyclyl, or heterocyclyl is independently optionally substituted.

The invention also relates to pharmaceutical compositions comprising the compounds and a pharmaceutically acceptable excipient and/or diluent, methods of treating a disease associated with ROCK2 and/or CK2 activity in a subject in need thereof comprising administering to the subject, such as a mammal, animal or human, a therapeutic amount of a compound of the invention, or a pharmaceutically acceptable salt thereof, or a pharmaceutical composition thereof. Such diseases can include obesity, metabolic disorder, energy homeostasis, immunological disorders, fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases.

The invention also includes the use of the compounds as a medicament or in the manufacture of a medicament.

The invention also relates to pharmaceutical compositions comprising the compounds and a pharmaceutically acceptable excipient and/or diluent, methods of treating a disease associated with AMP-activated protein kinase (AMPK) activity in a subject in need thereof comprising administering to the subject, such as a mammal, animal or human, a therapeutic amount of a compound of the invention, or a pharmaceutically acceptable salt thereof, or a pharmaceutical composition thereof. AMPK is a highly conserved sensor of low intracellular ATP levels that is rapidly activated after nearly all mitochondrial stresses, even those that do not disrupt the mitochondrial membrane potential. Activation of AMPK may provide a therapeutic benefit to treat conditions such as insulin resistance, metabolic syndrome, oxidative stress, inflammation and cancer.

The present invention includes pharmaceutical compositions comprising a substantially pure compound of the invention, or a pharmaceutically acceptable salt, stereoisomer, or hydrate thereof, and a pharmaceutically acceptable excipient and/or diluents.

### DETAILED DESCRIPTION

The present invention relates to compounds having the Formula (I):

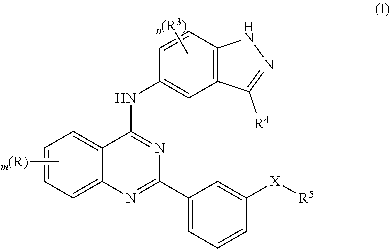

wherein the variables can be defined as above.

Preferred compounds have the formula:

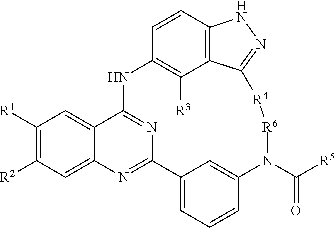

wherein R¹ and R² are independently selected from hydrogen, deuterium, halogen, methoxy or trifluoromethyloxy and the remaining variables can be defined as above.

Preferred compounds have the formula.

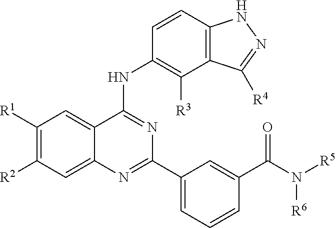

wherein the variables can be defined as above.

In preferred embodiments of the compound of any one of the preceding formulae, R⁶ is preferably hydrogen and R⁵ can be C₁₋₆ alkyl or C₁₋₆ haloalkyl. Preferably, R⁵ can be alkyl (such as methyl, ethyl, propyl, isopropyl, butyl, sec-butyl, t-butyl, pentyl or branched pentyl), fluoroalkyl (such as —CHF₂, —CH₂CHF₂, —CH₂CH₂CHF₂—, —CH(CH₃)CH₂CHF₂—, or CH₂CH₂CF₃), cycloalkyl-substituted alkyl (such as cyclopropylmethyl), or C₁₋₆ alkyl substituted by a —CONR⁸R⁹ group wherein R⁸ and R⁹ are each independently H, C₁₋₆ alkyl, C₁₋₆ haloalkyl, C₃₋₆ carbocyclyl, or 3-6 membered heterocyclyl, or, preferably, R⁸ and R⁹ are joined together to form an optionally substituted 3-6 membered heterocyclyl, such as

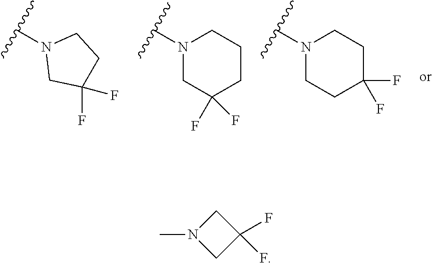

In preferred embodiments of the compound of any one of the preceding formulae, R⁶ is preferably hydrogen and R⁵ can optionally be substituted aryl or heteroaryl, such as phenyl. Preferred substituents include halogen, such as fluorine, or —CONR⁸R⁹ group wherein R⁸ and R⁹ are each independently H, C₁₋₆ alkyl, C₁₋₆ haloalkyl, C₃₋₆ carbocyclyl, or 3-6 membered heterocyclyl, or, preferably, R⁸ and R⁹ are joined together to form an optionally substituted 3-6 membered heterocyclyl, such as

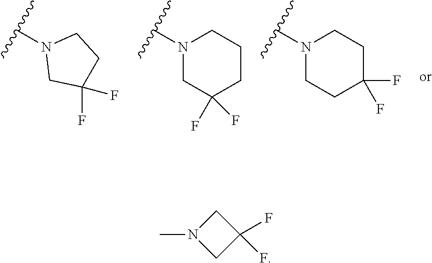

In preferred embodiments of the compound of any one of the preceding formulae, R⁶ can be H, preferably in combination with the preferred selections of R⁵.

Preferably, the compounds of the invention include but are not limited to inhibitors and activators of proteins and enzymes. Specifically, the compounds of the present invention may modulate the function of Rho-Kinase and/or CK2. The compounds of the invention may be useful in the treatment of obesity, metabolic disorder, energy homeostasis, immunological disorders, cancer, neuronal degeneration (peripheral or central), spinal cord injury, erectile dysfunction, atherosclerosis, hypertension, cerebral vasospasm, cerebral ischemia, restenosis, asthma, glaucoma, asthma, osteoporosis, fibrotic disease (liver and kidney), Kidney dialysis (epithelial stability), and neuronal degeneration inflammation.

The term “compound” means the chemical entities depicted in the chemical formula, salts, such as pharmaceutically acceptable salts, stereoisomers, including cis- and trans-isomers, R- and S-enantiomers, diastereomers, (D)-isomers, (L)-isomers, the racemic mixtures thereof, and other mixtures thereof. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “heteroatom” as used herein means an atom of any element other than carbon or hydrogen. Preferred heteroatoms are boron, nitrogen, oxygen, phosphorus, sulfur and selenium. Most preferred are nitrogen or oxygen.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C1-C30 for straight chain, C3-C30 for branched chain), and more preferably 20 or fewer. Preferred alkyls include methyl, ethyl, propyl, isopropyl, butyl, sec-butyl, and tert-butyl. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to six carbons, and more preferably from one to four carbon atoms. Likewise, “lower alkenyl” and “lower alkynyl” have similar chain lengths. Preferred alkyl groups are lower alkyls. In preferred embodiments, a substituent designated herein as alkyl is a lower alkyl.

The term “cycloalkyl” refers to saturated, carbocyclic groups having from 3 to 7 carbons in the ring. Preferred cycloalkyl groups include cyclopropyl, cyclobutyl, cyclopentyl and cyclohexyl.

The term “aralkyl”, as used herein, refers to an alkyl group substituted with an aryl group (e.g., an aromatic or heteroaromatic group). A preferred group is benzyl.

The terms “alkenyl” and “alkynyl” refer to unsaturated aliphatic groups analogous in length and possible substitution to the alkyls described above, but that contain at least one double or triple bond respectively. Preferred groups include vinyl, allyl and ethynyl.

Substituted alkyls, cycloalkyls, heterocyclyls, alkenyls, alkynyls and the like refers to substitution by independent replacement of one, two, or three or more of the hydrogen atoms with substituents including, but not limited to, —F, —Cl, —Br, —I, —OH, C1-C12-alkyl; C2-C12-alkenyl, C2-C12-alkynyl, —C3-C12-cycloalkyl, protected hydroxy, —NO2, —N3, —CN, —NH2, protected amino, oxo, thioxo, —NH-C1-C12-alkyl, —NH-C2-C8-alkenyl, —NH-C2-C8-alkynyl, —NH-C3-C12-cycloalkyl, —NH-aryl, —NH-heteroaryl, —NH-heterocycloalkyl, -dialkylamino, -diarylamino, -diheteroarylamino, —O-C1-C12-alkyl, —O-C2-C8-alkenyl, —O-C2-C8-alkynyl, —O-C3-C12-cycloalkyl, —O-aryl, —O-heteroaryl, —O-heterocycloalkyl, —C(O)—C1-C12-alkyl, —C(O)—C2-C8-alkenyl, —C(O)—C2-C8-alkynyl, —C(O)—C3-C12-cycloalkyl, —C(O)-aryl, —C(O)-heteroaryl, —C(O)-heterocycloalkyl, —CONH2, —CONH-C1-C12-alkyl, —CONH-C2-C8-alkenyl, —CONH-C2-C8-alkynyl, —CONH-C3-C12-cycloalkyl, —CONH-aryl, —CONH-heteroaryl, —CONH— heterocycloalkyl, —OCO2-C1-C12-alkyl, —OCO2-C2-C8-alkenyl, —OCO2-C2-C8-alkynyl, —OCO2-C3-C12-cycloalkyl, —OCO2-aryl, —OCO2-heteroaryl, —OCO2-heterocycloalkyl, —CO2-C1-C12 alkyl, —CO2-C2-C8 alkenyl, —CO2-C2-C8 alkynyl, CO2-C3-C12-cycloalkyl, —CO2- aryl, CO2-heteroaryl, CO2-heterocyloalkyl, —OCONH2, —OCONH-C1C12-alkyl, —OCONH-C2-C8-alkenyl, —OCONH-C2-C8-alkynyl, —OCONH-C3-C12-cycloalkyl, —OCONH-aryl, —OCONH— heteroaryl, —OCONH— heterocyclo-alkyl, —NHC(O)H, —NHC(O)—C1-C12-alkyl, —NHC(O)—C2-C8-alkenyl, —NHC(O)—C2-C8-alkynyl, —NHC(O)—C3-C12-cycloalkyl, —NHC(O)-aryl, —NHC(O)— heteroaryl, —NHC(O)-heterocyclo-alkyl, —NHCO2-C1-C12-alkyl, —NHCO2-C2-C8-alkenyl, —NHCO2-C2-C8-alkynyl, —NHCO2-C3-C12-cycloalkyl, —NHCO2-aryl, —NHCO2-heteroaryl, —NHCO2-heterocycloalkyl, —NHC(O)NH2, —NHC(O)NH-C1-C12-alkyl, —NHC(O)NH-C2-C8-alkenyl, —NHC(O)NH-C2-C8-alkynyl, —NHC(O)NH-C3-C12-cycloalkyl, —NHC(O)NH-aryl, —NHC(O)NH-heteroaryl, —NHC(O)NH-heterocycloalkyl, NHC(S)NH2, —NHC(S)NH-C1-C12-alkyl, —NHC(S)NH-C2-C8-alkenyl, —NHC(S)NH-C2-C8-alkynyl, —NHC(S)NH-C3-C12-cycloalkyl, —NHC(S)NH-aryl, —NHC(S)NH-heteroaryl, —NHC(S)NH-heterocycloalkyl, —NHC(NH)NH2, —NHC(NH)NH-C1-C12-alkyl, —NHC(NH)NH-C2-C8-alkenyl, —NHC(NH)NH-C2-C8-alkynyl, —NHC(NH)NH-C3-C12-cycloalkyl, —NHC(NH)NH-aryl, —NHC(NH)NH— heteroaryl, —NHC(NH)NH-heterocycloalkyl, —NHC(NH)—C1-C12-alkyl, —NHC(NH)—C2-C8-alkenyl, —NHC(NH)—C2-C8-alkynyl, —NHC(NH)—C3-C12-cycloalkyl, —NHC(NH)-aryl, —NHC(NH)-heteroaryl, —NHC(NH)-heterocycloalkyl, —C(NH)NH-C1-C12-alkyl, —C(NH)NH-C2-C8-alkenyl, —C(NH)NH-C2-C8-alkynyl, —C(NH)NH-C3-C12-cycloalkyl, —C(NH)NH-aryl, —C(NH)NH-heteroaryl, —C(NH)NH-heterocycloalkyl, —S(O)—C1-C12-alkyl, —S(O)—C2-C8-alkenyl, —S(O)—C2-C8-alkynyl, —S(O)—C3-C12-cycloalkyl, —S(O)-aryl, —S(O)-heteroaryl, —S(O)— heterocycloalkyl, —SO2NH2, —SO2NH-C1-C12-alkyl, —SO2NH-C2-C8-alkenyl, —SO2NH— C2-C8-alkynyl, —SO2NH-C3-C12-cycloalkyl, —SO2NH-aryl, —SO2NH-heteroaryl, —SO2NH-heterocycloalkyl, —NHSO2-C1-C12-alkyl, —NHSO2-C2-C8-alkenyl, —NHSO2-C2-C8-alkynyl, —NHSO2-C3-C12-cycloalkyl, —NHSO2-aryl, —NHSO2-heteroaryl, —NHSO2-heterocycloalkyl, —CH2NH2, —CH2SO2CH3, -aryl, -arylalkyl, -heteroaryl, -heteroarylalkyl, -heterocycloalkyl, —C3-C12-cycloalkyl, polyalkoxyalkyl, polyalkoxy, -methoxymethoxy, -methoxyethoxy, —SH, —S-C1-C12-alkyl, —S-C2-C8-alkenyl, —S-C2-C8-alkynyl, —S-C3-C12-cycloalkyl, —S-aryl, —S-heteroaryl, —S-heterocycloalkyl, or methylthio-methyl. In certain embodiments, the substituents are independently selected from halo, preferably Cl and F; C1-C4-alkyl, preferably methyl and ethyl; halo-C1-C4-alkyl, such as fluoromethyl, difluoromethyl, and trifluoromethyl; C2-C4-alkenyl; halo-C2-C4-alkenyl; C3-C6-cycloalkyl, such as cyclopropyl; C1-C4-alkoxy, such as methoxy and ethoxy; halo-C1-C4-alkoxy, such as fluoromethoxy, difluoromethoxy, and trifluoromethoxy; acetyl; —CN; —OH; NH2; C1-C4-alkylamino; di(C1-C4-alkyl)amino; and NO2. Preferably substituents do not reduce or adversely interfere with compound activity, such as can be predicted by chemical modeling. It is understood that the aryls, heteroaryls, alkyls, and the like can be further substituted. In some cases, each substituent in a substituted moiety is additionally optionally substituted with one or more groups, each group being independently selected from C1-C4-alkyl; —CF3, —OCH3, —OCF3, —F, —Cl, —Br, —I, —OH, —NO2, —CN, and —NH2. Preferably, a substituted alkyl group is substituted with one or more halogen atoms, more preferably one or more fluorine or chlorine atoms.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “aryl” as used herein includes 5- and 6-membered single-ring aromatic groups that may include from zero to four heteroatoms, for example, benzene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, aryls and/or heterocyclic groups.

The terms “heterocyclyl” or “heterocyclic group” refer to 3- to 10-membered ring structures, more preferably 5-, 6- or 7-membered rings, whose ring structures include one to four heteroatoms. Heterocycles can also be polycycles. Heterocyclic groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “polycyclyl” or “polycyclic group” refer to two or more rings (e.g., cycloalkyls, cycloalkenyls, aryls and/or heterocyclyls) in which two or more carbons are common to two adjoining rings, e.g., the rings are “fused rings”. Rings that are joined through non-adjacent atoms are termed “bridged” rings. Each of the rings of the polycyclic group can be substituted with such substituents as described above, for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

As used herein, the term “nitro” means —NO2; the term “halogen” or “halo” designates —F, —Cl, —Br or —I; the term “sulfhydryl” means —SH; the term “hydroxyl” means —OH; and the term “sulfonyl” means —SO2-.

The terms “amine” and “amino” are art-recognized and refer to both unsubstituted, substituted, primary, secondary, ternary and quaternary amines, e.g., a moiety that can be represented by the general formula —NRR′ or —N⁺RR′R″ where each of R, R′ and R″ can be H or an alkyl, alkenyl, alkynyl, aralkyl, aryl, and heterocyclic groups.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. The term lower alkoxy refers to an alkoxy group having from 1 to 6 carbon atoms.

The term “oxo” as used herein refers to an oxygen atom that has a double bond to a carbon.

The phrase “protecting group” as used herein means substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd ed.; Wiley: New York, 1991). Protecting groups can also be used to prepare prodrugs where the protecting group is pharmaceutically acceptable and physiologically labile.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

As used herein, the definition of each expression, e.g. alkyl, m, n, R, etc., when it occurs more than once in any structure, is intended to be independent of its definition elsewhere in the same structure.

The compounds of the invention can be made according to methods generally known in the art, as described, for example, in U.S. Pat. No. 8,357,693, which is incorporated by reference in its entirety. An example of a suitable synthetic scheme described therein is set forth below, maintaining the variables, as set forth therein which can be readily adapted to the variables used above.

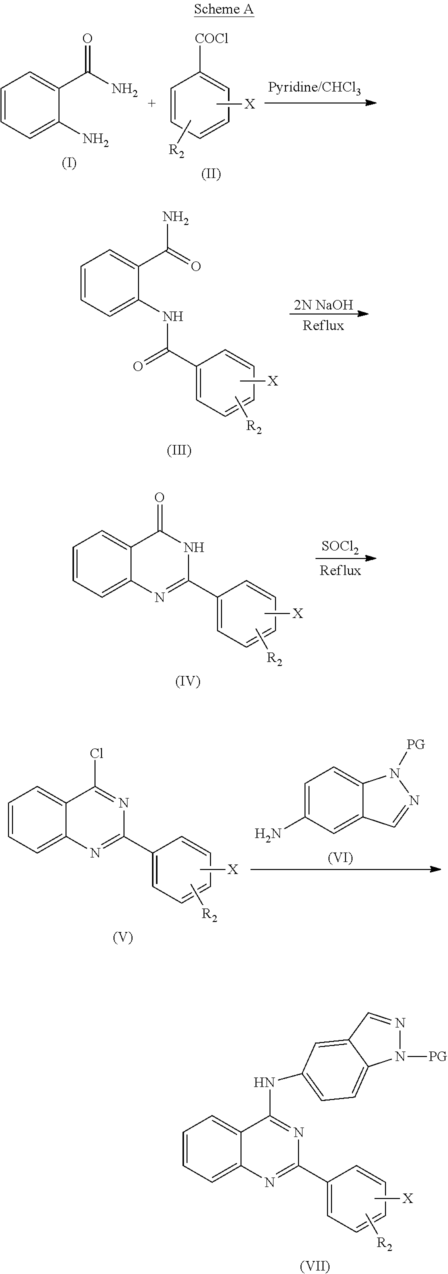

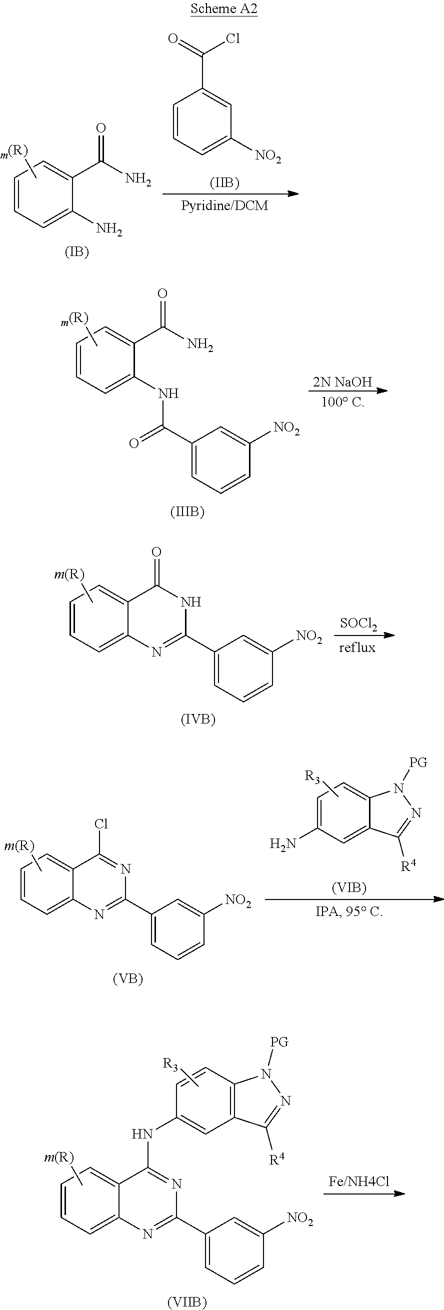

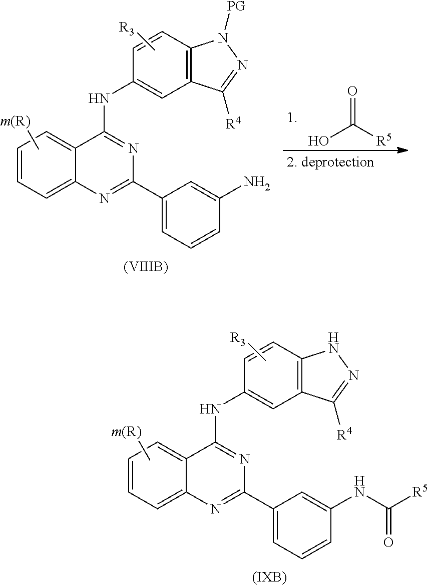

The general intermediate of formula (VII) or (VIIIB) may be prepared as illustrated in Scheme A and Scheme A2. As illustrated, anthralamide (2-aminobenzamide I or IB) is coupled with an appropriately substituted acid chloride of formula (II) or (IIB) in the presence of a base such as pyridine to give the benzamide (III or IIIB). The reaction is run in an aprotic solvent such as chloroform (CHCl3) or dichloromethane (DCM) at a temperature of −20 to 50° C., preferably at room temperature for 1-24 hours, preferably for 6 hours. Alternatively, the benzamide (III or IIIB) may be formed by treatment of the anthralamide (2-aminobenzamide (I or IB) with the benzoic acid in the presence of a coupling agent. Suitable coupling agents include N-cyclohexyl-N′-(4-diethylaminocyclohexyl)-carbodiimide (DCC), 1-(3-dimethylaminopropyl)-3-ethylcarbodiimide (EDC) and bromotripyrrolidino phosphonium hexafluorophosphate (PyBroP®), benzotriazolel-lyl-oxy-tris-pyrrolidino phosphonium hexafluorophosphate (PyBOP®) with suitable additives if necessary, which include 1-hydroxybenzotriazole (HOBt) and 3-hydroxy-4-oxo-3,4-dihydro-1,2,3-benzotriazine.

Cyclodehydration of compound (III) or (IIIB) is carried out under refluxing basic aqueous conditions using sodium hydroxide (NaOH) as base, though other bases such as potassium hydroxide (KOH) may also be used. The reaction is carried out at the reflux temperature of the mixture for about 1-24 hours, preferably about 4 hours. When X═OMe (compound VII) it may be necessary to exchange phenol protecting groups. This can be achieved via methods known to those skilled in the art.

The resulting quinazolin-4(3H)-one compound (IV or IVB) is aromatized to the chloroquinazoline (V or VB) by treatment with thionyl chloride (SOCl2) with catalytic dimethylformamide (DMF). The reaction mixture is heated to reflux for 1-6 hours preferably 4 hours. Alternatively, phosphorous oxy trichloride (POCl3) or oxalyl chloride can be used instead of SOCl2 to effect this transformation.

The chloroquinazoline (V or VB) is reacted with an appropriately protected 5-amino indazole (VI or VIB) to give the quinazoline (VII or VIIB). The reaction is carried out in iso-propanol at 95° C. for a reaction time of 30 minutes to 2 hours.

As illustrated in Scheme A2, reduction of nitro quinazoline (VIIB) is readily achieved using iron powder in aqueous ammonium chloride to provide the amino quinazoline (VIIIB). Subsequent derivatized by coupling with the appropriate carboxylic acid using a suitable coupling reagent provides compound (VIIIB). Alternatively, amino quinazoline (VIIIB) may be coupled with an appropriate acid chloride in aprotic solvent to provide compound (VIIIB). The final compound (IXB) is then obtained following deprotection of (VIIIB).

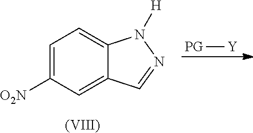

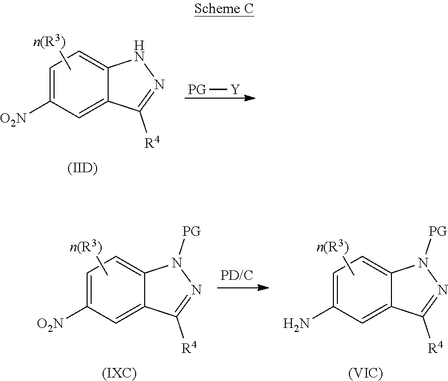

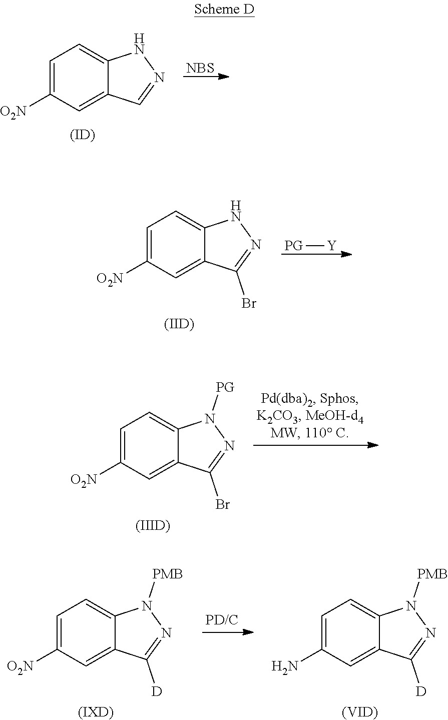

The protected indazole (VI, VIC and VID) can be prepared as depicted in Schemes B, C and D. The appropriate 5-Nitro-indazole (IIIV, IIIVC and IID) is suitably protected via methods known to those skilled in the art, preferably with a para-methoxybenzyl group or tert-butoxy carbonyl group.

PD-mediated deuteration of 3-bromo-5-nitro-indazole (IIID) with deuterium oxide provides 5-nitro-indazole-3-d (IXD).

The nitro group of compounds (IX), IXC) and (IXD) is reduced to the amino group via hydrogenation using a metal catalyst such as Pd/C in an inert solvent such as methanol (MeOH), 1,2 dimethoxethane (DME), ethanol (EtOH) or acetic acid (AcOH) or a combination of solvents preferably in a combination of MeOH and DME. The reaction can be carried out under balloon pressure or under a pressure of 20-50 pounds per square inch (p.s.i.).

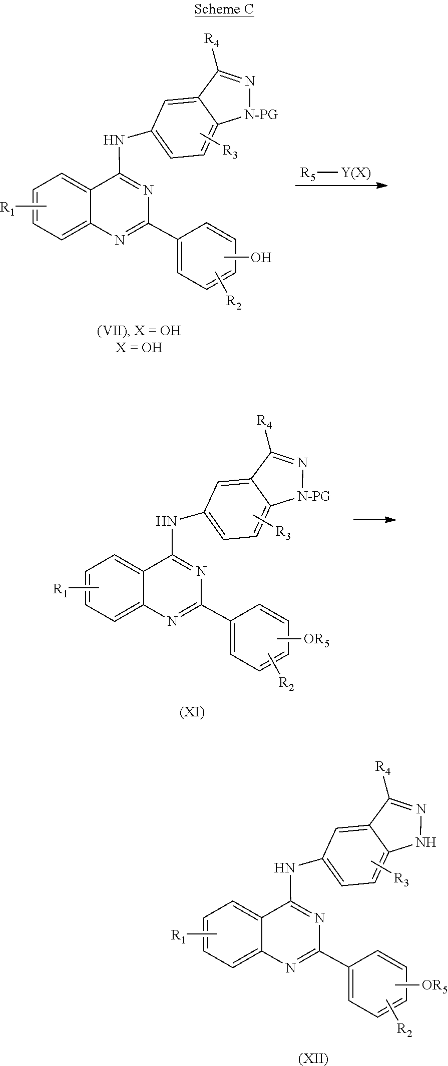

Compounds of formula (XII) can be synthesized as depicted in Scheme C. Compound (VII) can undergo selective deprotection of the O-protecting group functionality to give compound (VII) where X═OH. This can be done by a variety of methods, which are well known to those skilled in the art. The phenol (VII) is then alkylated with an electrophile of formula (X) in the presence of a base such as potassium carbonate (K2CO3), potassium tert-butoxide (KOtBu), sodium hydride (NaH), sodium hexamethylsilazide (NaHMDs) or potassium hexamethylsilazide (KHMDS) preferably K2CO3 to give the ether (XI). The reaction is run in an inert solvent such as DMF at a temperature of 20-100° C., preferably at 30-40° C. The electrophile (X) can be either a chloride (Y═Cl), bromide, (Y═Br), iodide (Y═I) or other suitable leaving group though it is preferred to use a bromide. Additives such as sodium iodide (NaI) or potassium iodide (KI) may be optionally added to the reaction.

Practitioners of the art will also recognize that the order of certain steps in the above schemes may be altered. Further, certain conditions such as solvent, temperature, etc. may be adjusted as would be recognized by the ordinarily skilled practitioner.

Reactive groups not involved in the above process steps can be protected with standard protecting groups during the reactions and removed by standard procedures (T. W. Greene & P. G. M. Wuts, Protecting Groups in Organic Synthesis, Third Edition, Wiley-Interscience) known to those of ordinary skill in the art. Presently preferred protecting groups include methyl, benzyl, acetate and tetrahydropyranyl for the hydroxyl moiety, and BOC, CBz, trifluoroacetamide and benzyl for the amino moiety, methyl, ethyl, tert-butyl and benzyl esters for the carboxylic acid moiety. The preferred protecting groups for the indazole moiety are BOC, CBz, trifluoroacetamide and benzyl.

In another aspect, the present invention provides a compound of the general formula I, wherein the compound is an inhibitor of Rho-kinase. Rho-kinase (ROCK), a serine/threonine kinase, serves as a target protein for small GTP-binding protein Rho. It serves as an important mediator of numerous cellular functions, including focal adhesions, motility, smooth muscle contraction, and cytokinesis. In smooth muscle, ROCK plays an important role in Ca2+ sensitization and the control of vascular tone. It modulates the level of phosphorylation of the myosin II light chain of myosin II, mainly through inhibition of myosin phosphatase, and contributes to agonist-induced Ca2+ sensitization in smooth muscle contraction.

Rho-kinase is found in two forms, ROCK 1 (ROCKβ; p160-ROCK) and ROCK 2 (ROCKα). Since for example a ROCK-mediated pathway plays an important role in vascular smooth muscle contraction, cell adhesion, and cell motility, it has gained importance in the pathogenesis of atherosclerosis. ROCK inhibitors are shown to suppress coronary artery spasms. A long-term inhibition of ROCK is reported to block the development of coronary arteriosclerotic lesions.

ROCK mediated pathways mediate numerous different cellular functions and ROCK inhibitors can be useful in treatments of patients in need thereof suffering from cardiovascular diseases such as hypertension, atherosclerosis, restenosis, cardiac hypertrophy, ocular hypertension, cerebral ischemia, cerebral vasospasm, penile erectile dysfunction, central nervous system disorders such as neuronal degeneration and spinal cord injury, and in neoplasias where inhibition of Rho-kinase has been shown to inhibit tumor cell growth and metastasis, angiogenesis, arterial thrombotic disorders such as platelet aggregation and leukocyte aggregation, asthma, regulation of intraoccular pressure, and bone resorption. Such treatment often relies on administering a therapeutic agent to a patient, wherein the therapeutic agent has a high specificity for a particular pathway or enzyme which is in need of regulation in the patient, by the therapeutic agent such as an enzyme inhibitor.

In one aspect of the present invention there is provided, a compound which is an inhibitor of a Rho-kinase (ROCK), preferably the compound of the present invention is an inhibitor of ROCK2.

The invention also relates to pharmaceutical compositions comprising the compounds and a pharmaceutically acceptable excipient and/or diluent, methods of treating a disease associated with ROCK2 and/or CK2 activity in a subject in need thereof comprising administering to the subject, such as a mammal, animal or human, a therapeutic amount of a compound of the invention, or a pharmaceutically acceptable salt thereof, or a pharmaceutical composition thereof. Such diseases can include obesity, metabolic disorder, energy homeostasis, immunological disorders, fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases. Preferably, the disease is obesity. Preferably, the disease is a metabolic disorder. Preferably, the disease is energy homeostasis. Preferably, the disease is an immunological disorder. Preferably, the disease is a fibrotic disease. Preferably, the disease is an inflammatory disease. Preferably, the disease is an autoimmune disease. Preferably, the disease is a cardiovascular disorder. Preferably, the disease is a central nervous system disorder. Preferably, the disease is a neoplastic disease. Preferably, the disease is a metabolic syndrome. Preferably, the disease is an ocular disease. Preferably, the disease is a renal disease. Preferably, the disease is a pulmonary disease. Preferably, the disease is muscular dystrophy. Preferably, the disease is sickle cell disease. Preferably, the disease is a viral disease.

The invention also relates to pharmaceutical compositions comprising the compounds and a pharmaceutically acceptable excipient and/or diluent, methods of treating a disease associated with AMP-activated protein kinase (AMPK) activity in a subject in need thereof comprising administering to the subject, such as a mammal, animal or human, a therapeutic amount of a compound of the invention, or a pharmaceutically acceptable salt thereof, or a pharmaceutical composition thereof. AMPK is a highly conserved sensor of low intracellular ATP levels that is rapidly activated after nearly all mitochondrial stresses, even those that do not disrupt the mitochondrial membrane potential. Activation of AMPK may provide a therapeutic benefit to treat conditions such as insulin resistance, metabolic syndrome, oxidative stress, inflammation and cancer.

The invention also includes the use of the compounds as a medicament or in the manufacture of a medicament.

Methods of determining kinase inhibition are well known in the art. For example, kinase activity of an enzyme and the inhibitory capacity of a test compound can be determined by measuring enzyme specific phosphorylation of a substrate. Commercial assays and kits can be employed. For example, kinase inhibition can be determined using an IMAP® assay (Molecular Devices). This assay method involves the use of a fluorescently-tagged peptide substrate. Phosphorylation of the tagged peptide by a kinase of interest promotes binding of the peptide to a trivalent metal-based nanoparticle via the specific, high affinity interaction between the phospho-group and the trivalent metal. Proximity to the nanoparticle results in increased fluorescence polarization. Inhibition of the kinase by a kinase inhibitor prevents phosphorylation of the substrate and thereby limits binding of the fluorescently-tagged substrate to the nanoparticle. Such an assay can be compatible with a microwell assay format, allowing simultaneous determination of IC50 of multiple compounds.

In another aspect of the present invention there is provided a method of treating a patient suffering from a disease comprising administering to a patient in need of such treatment a therapeutically effective amount of a compound of the present invention, wherein the disease is cardiovascular diseases such as hypertension, atherosclerosis, restenosis, cardiac hypertrophy, ocular hypertension, cerebral ischemia, cerebral vasospasm, penile erectile dysfunction, central nervous system disorders such as neuronal degeneration and spinal cord injury, and in neoplasias where inhibition of Rho-kinase has been shown to inhibit tumor cell growth and metastasis, angiogenesis, arterial thrombotic disorders such as platelet aggregation and leukocyte aggregation, asthma, regulation of intraoccular pressure, and bone resorption.

In another aspect, the present invention provides pharmaceutically acceptable compositions which comprise a therapeutically-effective amount of one or more of the compounds of the present invention, including but not limited to the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment, e.g. reasonable side effects applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, mesylate, ethane disulfonic, oxalic, isothionic, besylate and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel. Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug, prodrug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). The polymers used in the present invention have a significantly smaller molecular weight, approximately 100 daltons, compared to the large MW of 5000 daltons or greater that used in standard pegylation techniques. Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic® Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

Preferred compounds for use in the invention, including the pharmaceutical compositions and methods of treatment described above, can be selected from those in Table 1, and pharmaceutically acceptable salts thereof.

TABLE 1

| Example | Structure |
|---|---|
| 1 | 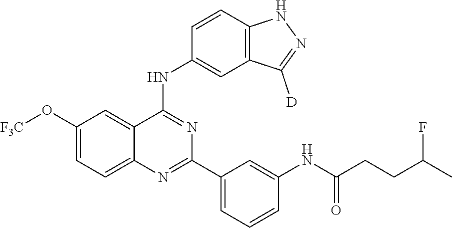 [SMILES: [2H]c1n[nH]c2ccc(Nc3nc(-c4cccc(NC(=O)CCC(C)F)c4)nc4ccc(OC(F)(F)F)cc34)cc12] |
| 2 | 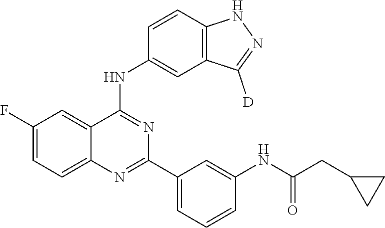 [SMILES: [2H]c1n[nH]c2ccc(Nc3nc(-c4cccc(NC(=O)CC5CC5)c4)nc4ccc(F)cc34)cc12] |
| 3 | 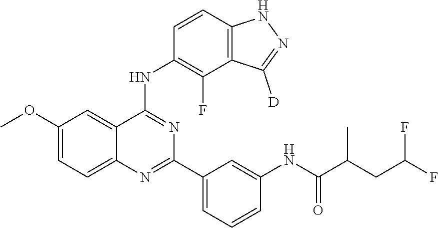 [SMILES: [2H]c1n[nH]c2ccc(Nc3nc(-c4cccc(NC(=O)C(C)CC(F)F)c4)nc4ccc(OC)cc34)c(F)c12] |
| 4 | 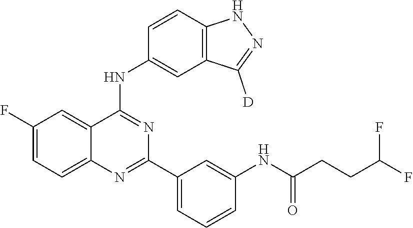 [SMILES: [2H]c1n[nH]c2ccc(Nc3nc(-c4cccc(NC(=O)CCC(F)F)c4)nc4ccc(F)cc34)cc12] |
| 5 | 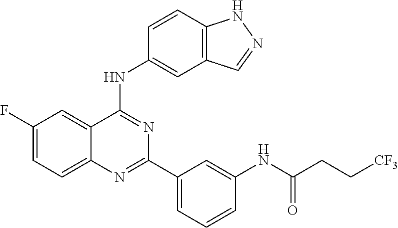 [SMILES: O=C(CCC(F)(F)F)Nc1cccc(-c2nc(Nc3ccc4[nH]ncc4c3)c3cc(F)ccc3n2)c1] |
| 6 | 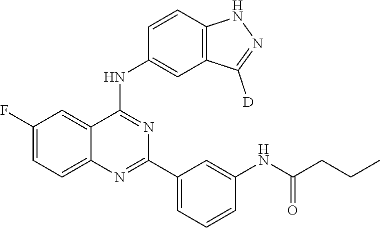 [SMILES: [2H]c1n[nH]c2ccc(Nc3nc(-c4cccc(NC(=O)CCC)c4)nc4ccc(F)cc34)cc12] |
| 7 | 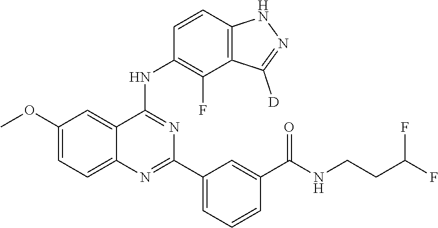 [SMILES: [2H]c1n[nH]c2ccc(Nc3nc(-c4cccc(C(=O)NCCC(F)F)c4)nc4ccc(OC)cc34)c(F)c12] |
| 8 | 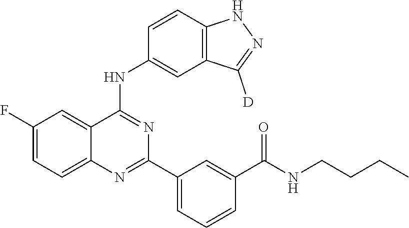 [SMILES: [2H]c1n[nH]c2ccc(Nc3nc(-c4cccc(C(=O)NCCCC)c4)nc4ccc(F)cc34)cc12] |
| 9 | 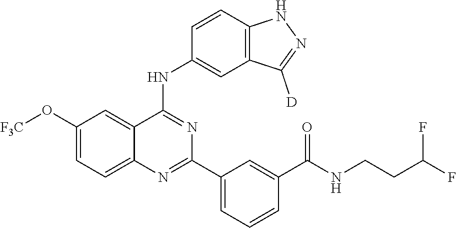 [SMILES: [2H]c1n[nH]c2ccc(Nc3nc(-c4cccc(C(=O)NCCC(F)F)c4)nc4ccc(OC(F)(F)F)cc34)cc12] |
| 10 | 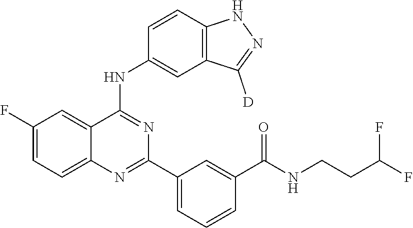 [SMILES: [2H]c1n[nH]c2ccc(Nc3nc(-c4cccc(C(=O)NCCC(F)F)c4)nc4ccc(F)cc34)cc12] |
| 11 | 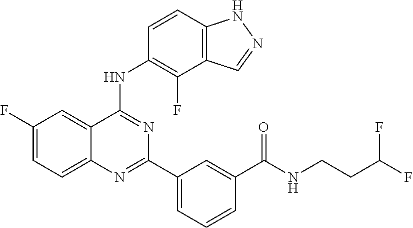 [SMILES: O=C(NCCC(F)F)c1cccc(-c2nc(Nc3ccc4[nH]ncc4c3F)c3cc(F)ccc3n2)c1] |
| 12 | 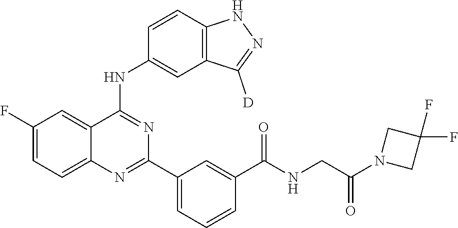 [SMILES: [2H]c1n[nH]c2ccc(Nc3nc(-c4cccc(C(=O)NCC(=O)N5CC(F)(F)C5)c4)nc4ccc(F)cc34)cc12] |
| 13 | 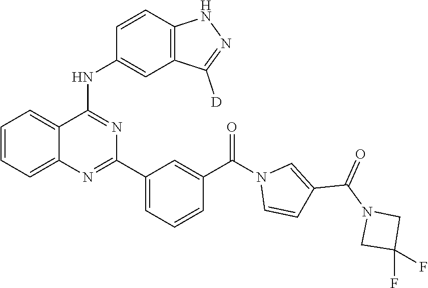 [SMILES: [2H]c1n[nH]c2ccc(Nc3nc(-c4cccc(C(=O)n5ccc(C(=O)N6CC(F)(F)C6)c5)c4)nc4ccccc34)cc12] |
| 14 | 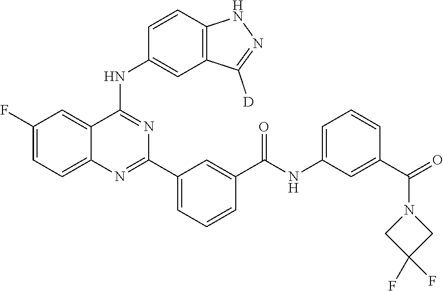 [SMILES: [2H]c1n[nH]c2ccc(Nc3nc(-c4cccc(C(=O)Nc5cccc(C(=O)N6CC(F)(F)C6)c5)c4)nc4ccc(F)cc34)cc12] |
| 15 | 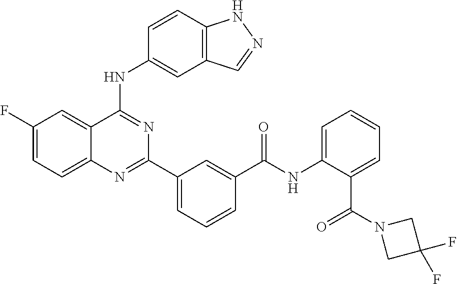 [SMILES: O=C(Nc1ccccc1C(=O)N1CC(F)(F)C1)c1cccc(-c2nc(Nc3ccc4[nH]ncc4c3)c3cc(F)ccc3n2)c1] |
| 16 | 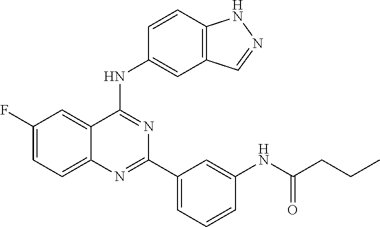 [SMILES: CCCC(=O)Nc1cccc(-c2nc(Nc3ccc4[nH]ncc4c3)c3cc(F)ccc3n2)c1] |
| 17 | 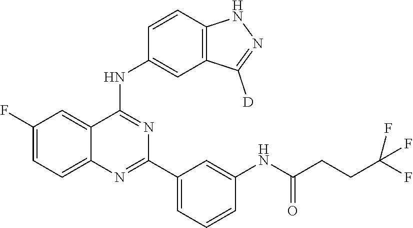 [SMILES: [2H]c1n[nH]c2ccc(Nc3nc(-c4cccc(NC(=O)CCC(F)(F)F)c4)nc4ccc(F)cc34)cc12] |
| 18 | 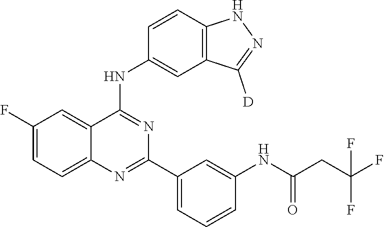 [SMILES: [2H]c1n[nH]c2ccc(Nc3nc(-c4cccc(NC(=O)CC(F)(F)F)c4)nc4ccc(F)cc34)cc12] |
| 19 | 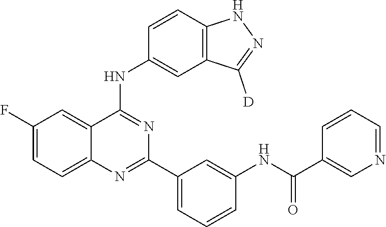 [SMILES: [2H]c1n[nH]c2ccc(Nc3nc(-c4cccc(NC(=O)c5cccnc5)c4)nc4ccc(F)cc34)cc12] |
| 20 | 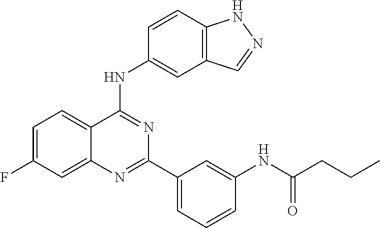 [SMILES: CCCC(=O)Nc1cccc(-c2nc(Nc3ccc4[nH]ncc4c3)c3ccc(F)cc3n2)c1] |
| 21 | 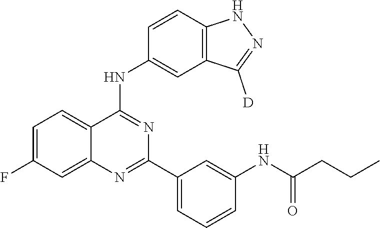 [SMILES: [2H]c1n[nH]c2ccc(Nc3nc(-c4cccc(NC(=O)CCC)c4)nc4cc(F)ccc34)cc12] |
| 22 | 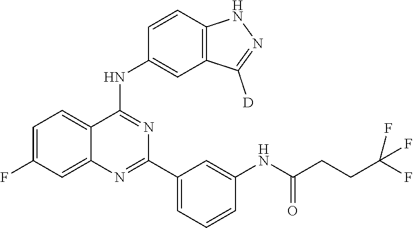 [SMILES: [2H]c1n[nH]c2ccc(Nc3nc(-c4cccc(NC(=O)CCC(F)(F)F)c4)nc4cc(F)ccc34)cc12] |
| 23 | 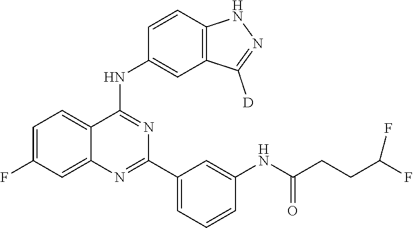 [SMILES: [2H]c1n[nH]c2ccc(Nc3nc(-c4cccc(NC(=O)CCC(F)F)c4)nc4cc(F)ccc34)cc12] |
| 24 | 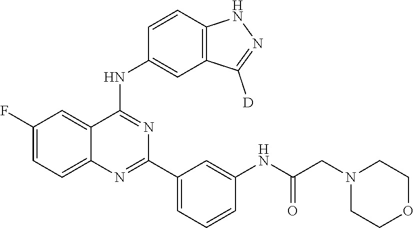 [SMILES: [2H]c1n[nH]c2ccc(Nc3nc(-c4cccc(NC(=O)CN5CCOCC5)c4)nc4ccc(F)cc34)cc12] |
| 25 | 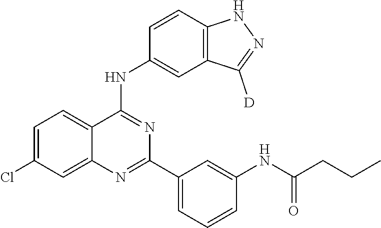 [SMILES: [2H]c1n[nH]c2ccc(Nc3nc(-c4cccc(NC(=O)CCC)c4)nc4cc(Cl)ccc34)cc12] |
| 26 | 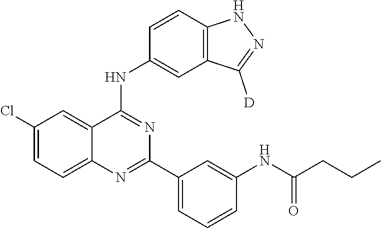 [SMILES: [2H]c1n[nH]c2ccc(Nc3nc(-c4cccc(NC(=O)CCC)c4)nc4ccc(Cl)cc34)cc12] |
| 27 | 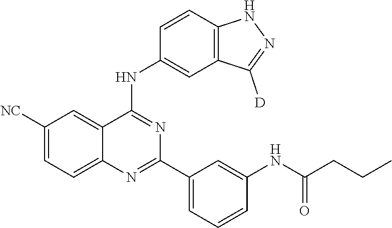 [SMILES: [2H]c1n[nH]c2ccc(Nc3nc(-c4cccc(NC(=O)CCC)c4)nc4ccc(C#N)cc34)cc12] |
| 28 | 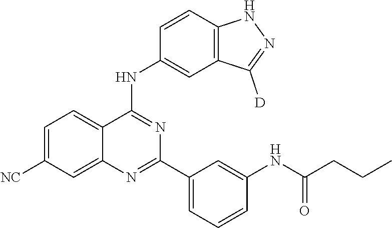 [SMILES: [2H]c1n[nH]c2ccc(Nc3nc(-c4cccc(NC(=O)CCC)c4)nc4cc(C#N)ccc34)cc12] |
| 29 | 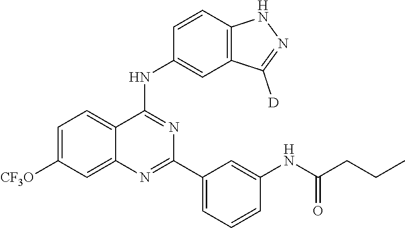 |
| 30 | 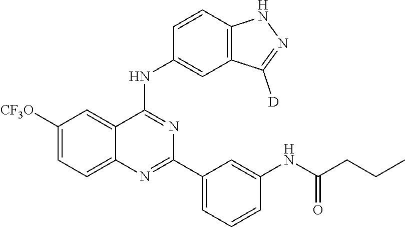 |

### EXAMPLES

The following examples are intended to illustrate the invention and are not intended to limit it.

### Example 1—Compound Syntheses

### General Abbreviation Definitions

ACN acetonitrile
aq. aqueous
DCM dichloromethane
DIPEA diisopropylethylamine
DMF Dimethylforamide
DMSO dimethyl sulfoxide
Et₂O diethyl ether
EtOAc ethyl acetate
h hour
HCl hydrochloric aid
MeOH methanol
MS mass spectrometry
m/z mass to charge ratio
MW microwave
NaOH sodium hydroxide
NBS N-bromosuccinimide
RT room temperature
Pet ether petroleum ether
PMB para-methoxybenzyl
T3P propylphosphonic anhydride
TEA triethyl amine
TFA trifluoroacetic acid
TLC thin layer chromatography

### Synthesis of Intermediate 3-bromo-5-nitro-1H-indazole

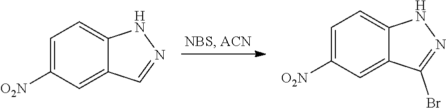 [SMILES: O=[N+]([O-])c1ccc2[nH]ncc2c1 ; O=[N+]([O-])c1ccc2[nH]nc(Br)c2c1]

A solution of 5-nitro-1H-indazole (10 g, 61.3 mmol) in acetonitrile (100 mL) was cooled to 0° C. under N₂ atmosphere and NBS (12.002 g, 67.431 mmol) was added slowly portion wise. The reaction mixture was stirred at RT for 16 h as progress of the reaction was monitored by TLC (20% ethyl acetate:pet ether). After completion of the reaction, the reaction mixture was quenched with ice-cooled water (100 mL) and extracted with ethyl acetate (100 mL×3). The combined organic extracts were washed successively with water (200 mL) and brine solution (200 mL) and then dried over anhydrous Na₂SO₄ and filtered. The solvent was evaporated under reduced pressure to obtain crude product which was triturated with pet ether (100 mL) and dried under vacuum to deliver product 3-bromo-5-nitro-1H-indazole (11 g) as a pale brown solid. 1H NMR (400 MHz, DMSO-d6) δ ppm: 14.10 (s, 1H), 8.50 (d, J=9.2 Hz, 1H), 7.81 (d, J=9.2 Hz, 1H).

### Synthesis of Intermediate 3-bromo-1-(4-methoxybenzyl)-5-nitro-1H-indazole

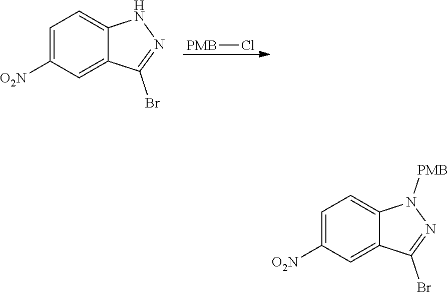

To a solution of 3-bromo-5-nitro-1H-indazole (13.5 g, 55.8 mmol) in DMF (130 mL) cooled to 0° C. was added potassium carbonate (23.1 g, 167.3 mmol). The resulting reaction mixture was stirred for 15 min followed by the addition of 1-(chloromethyl)-4-methoxybenzene (11.4 mL, 83.7 mmol) at 0° C. The resulting reaction mixture was stirred at RT for 2 h as progress of the reaction was monitored by TLC (20% ethyl acetate:pet ether). The reaction mixture was quenched with ice cooled water (50 mL). The resulting precipitated solid was filtered and washed with pet ether (100 mL) to deliver 3-bromo-1-(4-methoxybenzyl)-5-nitro-1H-indazole as an off-white solid (12 g). 1H NMR (400 MHz, DMSO-d6) δ ppm: 8.48 (s, 1H), 8.35 (d, J=9.2 Hz, 1H), 8.09 (d, J=9.2 Hz, 1H), 7.28 (d, J=8.4 Hz, 1H), 6.90 (d, J=8.8 Hz, 1H), 5.67 (s, 2H), 3.71 (s, 3H).

### Synthesis of Intermediate 1-(4-methoxybenzyl)-5-nitro-1H-indazole-3-d

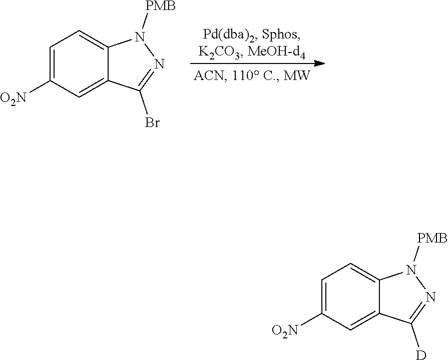

In a Microwave vial, charged 3-bromo-1-(4-methoxybenzyl)-5-nitro-1H-indazole (1 g, 2.8 mmol) in acetonitrile (5 mL), was added Pd(dba)₂ (0.159 g, 0.276 mmol), potassium carbonate anhydrous (0.763 g, 5.522 mmol), S-phos (0.340 g, 0.828 mmol) and MeOH-d₄ (5 mL). The reaction mixture was stirred at 110° C. for 2 h under microwave conditions as progress of the reaction was monitored by TLC (20% ethyl acetate:pet ether). Upon completion, the reaction was cooled and concentrated under reduced pressure. The crude residue was directly charged on a silica gel (100-200 mesh) column and the product was eluted with 15% ethyl acetate in pet ether to deliver 1-(4-methoxybenzyl)-5-nitro-1H-indazole-3-d (0.5 g) as a pale-yellow solid. 1H NMR (400 MHz, DMSO-d6) δ ppm: 8.83 (s, 1H), 8.23 (d, J=9.2 Hz, 1H), 7.97 (d, J=9.2 Hz, 1H), 7.25 (d, J=8.4 Hz, 1H), 6.88 (d, J=6.8 Hz, 1H), 5.67 (s, 2H), 3.71 (s, 3H).

### Synthesis of Intermediate 1-(4-methoxybenzyl)-1H-indazol-3-d-5-amine

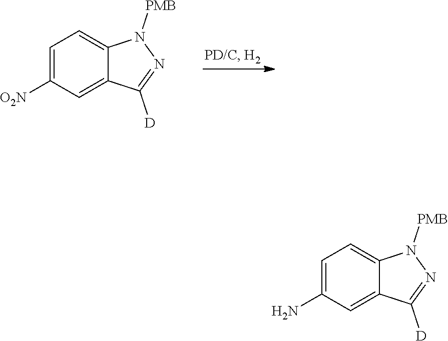

To a solution of 1-(4-methoxybenzyl)-5-nitro-1H-indazole-3-d (0.45 g, 1.583 mmol) in methanol (5 mL) and ethyl acetate (5 mL) was added 10% Palladium on Carbon (wetted with ca. 55% Water) (50 mg) and the reaction was stirred for 2 hours under Hydrogen atmosphere (balloon) as progress of the reaction was monitored by TLC (30% ethyl acetate in Pet ether). The reaction mixture was diluted with ethyl acetate (25 mL) and filtered through celite. The celite bed was washed with ethyl acetate (25 mL) and the collected filtrate was concentrated to give crude product. The crude solid was washed with Pet Ether to afford desired product 1-(4-methoxybenzyl)-1H-indazol-3-d-5-amine (0.3 g) as a pale red solid. 1H NMR (400 MHz, DMSO-d6) δ ppm: 7.35 (d, J=8.8 Hz, 1H), 7.16 (d, J=8.4 Hz, 2H), 6.84 (d, J=8.8 Hz, 2H), 6.78 (m, 2H), 5.42 (s, 2H), 3.68 (s, 3H).

### Synthesis of Intermediate N-(2-carbamoyl-4-fluorophenyl)-3-nitrobenzamide

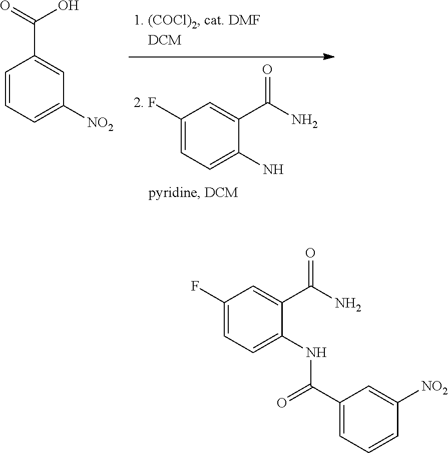

To a cooled solution of 3-nitrobenzoic acid (12 g, 0.0718 moles) in DCM (120 mL) was added oxalyl chloride (18.4 mL, 0.2155 moles) and DMF (cat) at 0° C., under N₂ atmosphere. The reaction mixture was allowed to stir at ambient temperature for 30 min, as progress of the reaction was monitored by TLC (50% ethyl acetate in pet ether). The reaction mixture was concentrated under pressure to deliver 3-nitrobenzoyl chloride (13.9 g). In a separate flask, a cooled solution of 2-amino-5-fluorobenzamide (11 g, 0.0714 moles) in DCM (110 mL) was prepared at 0° C. under N₂ atmosphere and then pyridine (11.5 mL, 0.142 moles) was added and the resulting solution was allowed to stir for 30 min. To this reaction mixture was added a solution of 3-nitrobenzoyl chloride (13.242 g, 0.0714 mmol) in DCM (50 mL) via dropwise addition at 0° C. The resulting reaction was stirred at ambient temperature for 30 min as progress was monitored by TLC (50% ethyl acetate in pet ether). The reaction was quenched with ice cold water, and the resulting precipitate was collected by filtration. The solid was subsequently washed with water and hexane and then dried under vacuum to obtain N-(2-carbamoyl-4-fluorophenyl)-3-nitrobenzamide (20 g) as an off-white solid. 1H NMR (400 MHz, DMSO-d6) δ ppm: 13.00 (s, 1H), 8.72 (t, J=1.90 Hz, 1H), 8.61-8.69 (m, 1H), 8.48 (ddd, J=8.22, 2.23, 0.92 Hz, 2H), 8.29-8.41 (m, 1H), 8.06 (br s, 1H), 7.77-7.94 (m, 2H), 7.50 (ddd, J=9.14, 8.10, 2.93 Hz, 1H).

### Synthesis of Intermediate 6-fluoro-2-(3-nitrophenyl)quinazolin-4(3H)-one

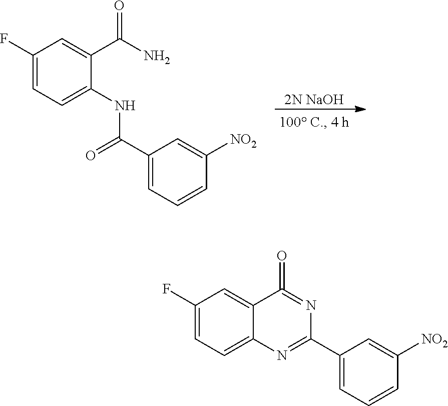

A solution of N-(2-carbamoyl-4-fluorophenyl)-3-nitrobenzamide (10 g, 0.033 moles) and 2N Sodium hydroxide solution (150 mL) was heated to 100° C. for 4 has reaction progress was monitored by TLC (50% ethyl acetate in pet ether). The resulting precipitate was collected by filtration and washed with pet ether (50 mL), co-distilled with toluene (100 mL) and dried under vacuum to provide product 6-fluoro-2-(3-nitrophenyl)quinazolin-4(3H)-one (8.5 g) as an off-white solid. 1H NMR (400 MHz, DMSO-d6) δ ppm: 9.18-9.22 (m, 1H), 8.83 (dt, J=7.89, 1.25 Hz, 1H), 8.23 (ddd, J=8.10, 2.48, 1.04 Hz, 1H), 7.70 (t, J=7.95 Hz, 1H), 7.62 (dd, J=9.41, 3.06 Hz, 1H), 7.57 (dd, J=8.99, 5.20 Hz, 1H), 7.36 (td, J=8.80, 3.06 Hz, 1H); Mass (ESI): m/z 286.1724 [M]+.

### Synthesis of Intermediate 4-chloro-6-fluoro-2-(3-nitrophenyl)quinazoline

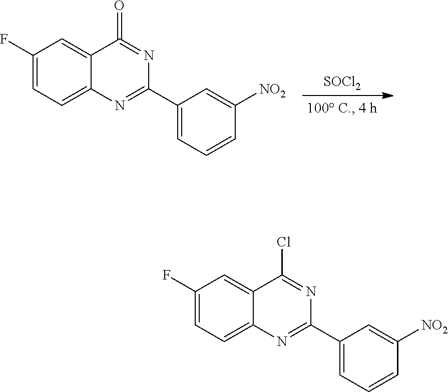

To 6-fluoro-2-(3-nitrophenyl)quinazolin-4(3H)-one (8.5 g, 0.029 moles) and added SOCl₂ (85 mL) and DMF (catalytic) at 0° C. under N₂ atmosphere. The reaction mixture was heated to 100° C. for 4 h and monitored by TLC (30% ethyl acetate in pet ether). The reaction mixture was concentrated under vacuum and quenched with ice water. The resulting precipitate was filtered, washed with water, co-distilled with toluene, and washed with per ether to obtain product 4-chloro-6-fluoro-2-(3-nitrophenyl)quinazoline (8 g).

### Synthesis of Intermediate 6-fluoro-N-(1-(4-methoxybenzyl)-1H-indazol-5-yl-3-d)-2-(3-nitrophenyl)quinazolin-4-amine

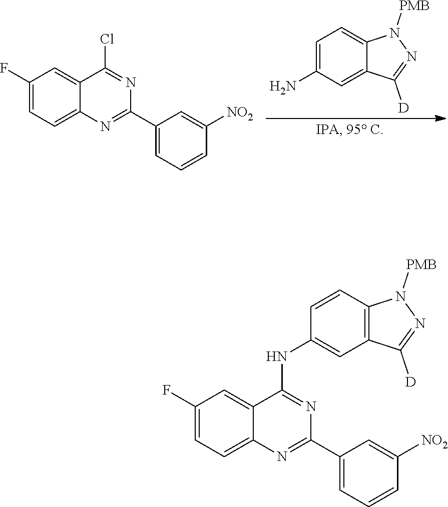

To a stirred solution of 4-chloro-6-fluoro-2-(3-nitrophenyl)quinazoline (300 mg, 0.99 mmol) in 2-propanol (60 mL) was added 1-(4-methoxybenzyl)-1H-indazol-3-d-5-amine (276.35 mg, 1.09 mmol) and the resulting mixture was stirred at 95° C. for 2 hours as the reaction progress was monitored by TLC (50% ethyl acetate in pet ether). The reaction mixture was filtered and the solid was washed with diethyl ether and dried under vacuum to afford product 6-fluoro-N-(1-(4-methoxybenzyl)-1H-indazol-5-yl-3-d)-2-(3-nitrophenyl)quinazolin-4-amine (450 mg). ¹H NMR (400 MHz, DMSO-d₆) δ ppm: 10.45 (s, 1H), 9.13-9.16 (m, 1H), 8.74 (d, J=7.44 Hz, 1H), 8.52 (dd, J=9.76, 2.75 Hz, 1H), 8.38 (ddd, J=8.22, 2.41, 1.00 Hz, 1H), 8.34-8.36 (m, 1H), 8.17 (s, 1H), 8.04 (dd, J=9.13, 5.38 Hz, 1H), 7.77-7.90 (m, 4H), 7.29 (d, J=8.75 Hz, 2H), 6.87-6.92 (m, 2H), 5.62 (s, 2H), 3.71 (s, 3H)

### Synthesis of Intermediate 2-(3-aminophenyl)-6-fluoro-N-(1-(4-methoxybenzyl)-1H-indazol-5-yl-3-d)quinazolin-4-amine

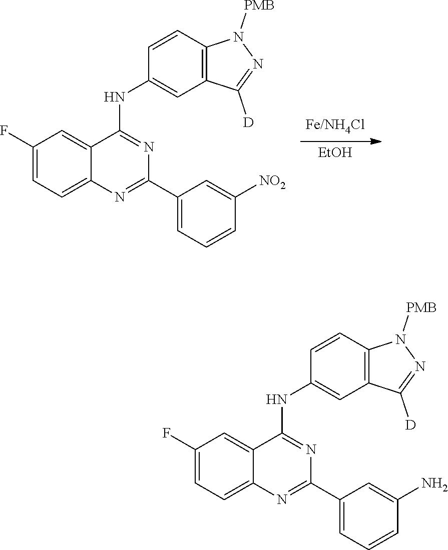

To a solution of 6-fluoro-N-(1-(4-methoxybenzyl)-1H-indazol-5-yl-3-d)-2-(3-nitrophenyl)quinazolin-4-amine (800 mg, 1.54 mmol) in ethanol (30 mL) and water (4 mL) was added iron powder (514.38 mg, 9.21 mmol) and ammonium chloride (328.4 g, 6.14 mmol). The resulting mixture was then stirred at 95° C. for 4 h as progress of the reaction was monitored by TLC (50% ethyl acetate in pet ether) and LCMS. The reaction mixture was filtered through celite, and the celite bed was washed with 100 mL of 10% MeOH in DCM. The filtrate was concentrated under vacuum to provide crude solid that was subsequently triturated with diethyl ether (20 mL) and dried under vacuum to afford 2-(3-aminophenyl)-6-fluoro-N-(1-(4-methoxybenzyl)-1H-indazol-5-yl-3-d)quinazolin-4-amine (620 mg).

### Synthesis of Intermediate 4,4-difluoro-N-(3-(6-fluoro-4-((1-(4-methoxybenzyl)-1H-indazol-5-yl-3-d)amino)quinazolin-2-yl)phenyl)butanamide

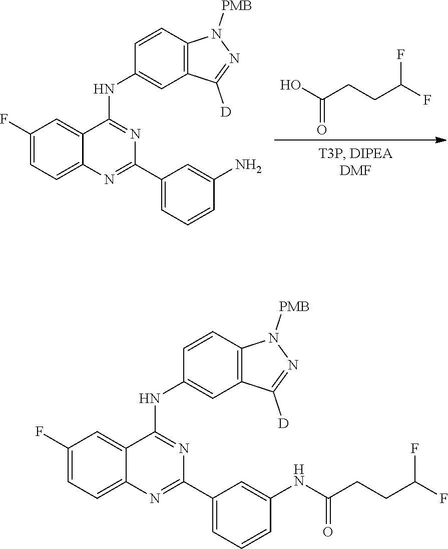

To a solution of 2-(3-aminophenyl)-6-fluoro-N-(1-(4-methoxybenzyl)-1H-indazol-5-yl-3-d)quinazolin-4-amine (150 mg, 0.305 mmol) in DMF (5 mL) was added 4,4-difluorobutanoic acid (56.8 mg, 0.46 mmol), DIPEA (0.152 mL, 0.916 mmol) and propylphosphonic anhydride (0.115 mL, 0.397 mmol) and the mixture was stirred at room temperature for 16 hours. The reaction was quenched with ice water and the precipitated solid was collected, triturated with diethyl ether and dried under vacuum to afford product 4,4-difluoro-N-(3-(6-fluoro-4-((1-(4-methoxybenzyl)-1H-indazol-5-yl-3-d)amino)quinazolin-2-yl)phenyl)butanamide (110 mg).

### Example 4: Synthesis of N-(3-(4-((1H-indazol-5-yl-3-d)amino)-6-fluoroquinazolin-2-yl)phenyl)-4,4-difluorobutanamide

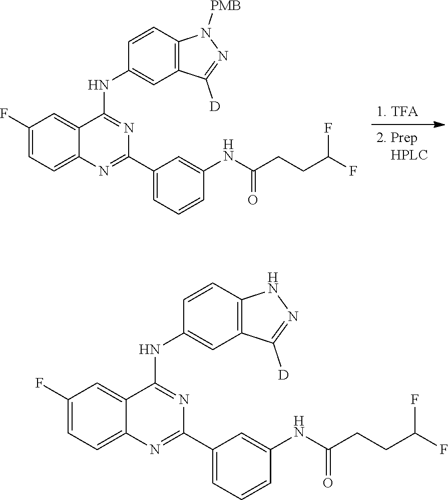

A solution of 4,4-difluoro-N-(3-(6-fluoro-4-((1-(4-methoxybenzyl)-1H-indazol-5-yl-3-d)amino) quinazolin-2-yl)phenyl)butanamide (80 mg, 0.134 mmol) was stirred in TFA (0.5 M in water) under microwave conditions for 2 hours at 110° C. The reaction was concentrated under vacuum, basified with saturated NaHCO₃ solution (10 mL) and extracted with ethyl acetate (2×10 mL). The combined extracts were dried over sodium sulphate and concentrated under reduced pressure. The crude product (80 mg) was purified by preparative HPLC using a YMC-C18 [150×20 mm]5μ column and gradient method (mobile phase A: 0.1% HCl in water; mobile phase B: acetonitrile) to provide desired product N-(3-(4-((1H-indazol-5-yl-3-d)amino)-6-fluoroquinazolin-2-yl)phenyl)-4,4-difluorobutanamide as an HCL salt (32 mg). Mass (ESI): m/z 478.20 [M]+, 1H NMR (400 MHz, DMSO-d6) δ ppm: 10.31 (s, 1H), 8.84 (s, 1H), 8-76-8.68 (d, 1H), 8.40 (s, 1H), 8.11 (m, 1H), 8.04-8.02 (d, 1H), 7.95-7.91 (m, 1H), 7.87-7.84 (dd, 1H) 7.77-7.65 (m, 2H), 7.53-7.49 (m, 1H), 6.35-6.06 (m, 1H), 2.59-2.55 (m, 2H), 2.28-2.14 (m, 2H).

Examples of the invention are prepared following similar methodology from the appropriately substituted 2-aminobenzamide, 1H-indazol-5-amine and carboxylic acid.

| | Example | Mass | |
|---|---|---|---|
| Structure | No. | (ESI): m/z | 1H NMR (DMSO-d₆, δ ppm) |
| 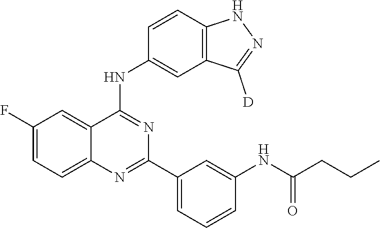 [SMILES: [2H]c1n[nH]c2ccc(Nc3nc(-c4cccc(NC(=O)CCC)c4)nc4ccc(F)cc34)cc12] | 6 | 442.35 [M]+ | 10.23 (s, 1H), 8.84-8.80 (m, 2H), 8.41 (s, 1H), 8.27-8.22 (m, 1H), 8.04-8.02 (m, 1H), 8.0-7.96 (m, 1H), 7.89-7.88 (d, 1H), 7.87-7.86 (m, 1H), 7.69-7.68 (m, 2H), 7.54-7.51 (m, 1H), 2.37-2.35 (m, 2H), 1.71-1.65 (m, 2H), 0.97-0.94 (t, 3H) |
| 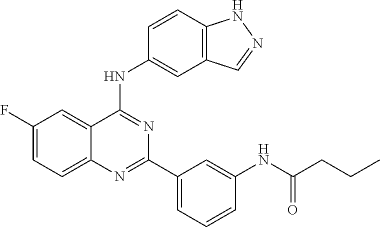 [SMILES: CCCC(=O)Nc1cccc(-c2nc(Nc3ccc4[nH]ncc4c3)c3cc(F)ccc3n2)c1] | 16 | 441.40 [M]+ | 10.12 (s, 1H), 8.80 (S, 1H), 8.75-8.72 (m, 1H), 8.42 (s, 1H), 8.27 (s, 1 H), 8.14 (m, 1H), 8.02-8.00 (d, 1H), 7.97- 7.93 (m1H), 7.90-7.89 (m, 1H), 7.88- 7.85 (dd, 1H), 7.70-7.68 (d, 2H), 7.53- 7.50 (m, 1H), 2.38-2.34 (m, 2H), 1.73- 1.64 (m, 2H), 0.98-0.96 (t, 3H) |
| 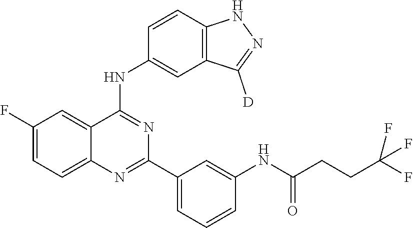 [SMILES: [2H]c1n[nH]c2ccc(Nc3nc(-c4cccc(NC(=O)CCC(F)(F)F)c4)nc4ccc(F)cc34)cc12] | 17 | 496.35 [M]+ | 10.39 (S, 1H), 8.76 (S, 1H), 8.70-8.68 (m, 1H), 8.39 (S, 1H), 8.10-8.02 (m, 2H), 7.95-7.90 (m, 1H), 7.87-7.84 (dd, 1 H), 7.71-7.68 (m, 2H), 7.55-5.51 (m, 1H), 2.68-2.61 (m, 4H) |
| 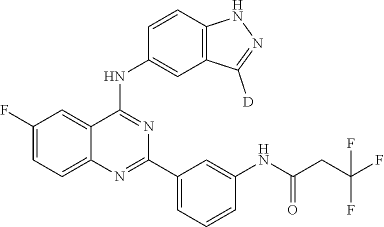 [SMILES: [2H]c1n[nH]c2ccc(Nc3nc(-c4cccc(NC(=O)CC(F)(F)F)c4)nc4ccc(F)cc34)cc12] | 18 | 482.20 [M]+ | 10.92-10.74 (m, 2H), 8.77-8.74 (m, 1H), 8.43 (m, 1H), 8.15 (m, 1H), 8.11- 8.09 (d, 1H), 7.96-7.92 (m, 1H), 7.88- 7.86 (dd, 1H), 7.73-7.67 (m, 2H), 7.58-7.54 (m, 1H), 3.66-3.60 (m, 2H) |
| 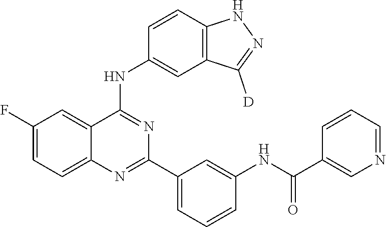 [SMILES: [2H]c1n[nH]c2ccc(Nc3nc(-c4cccc(NC(=O)c5cccnc5)c4)nc4ccc(F)cc34)cc12] | 19 | 47.24 [M]+ | 10.88 (s, 1H), 9.27 (s, 1H), 8.98 (m, 1H), 8.89-8.88 (d, 1H), 8.80-8.78 (m, 1H), 8.555-8.53 (m, 1H), 8.41 (s, 1H), 8.24-8.21 (m, 1H), 8.17-8.15 (m, 1H), 8.00-7.96 (m, 1H), 7.93-7.88 (m, 2H), 7.78-7.75 (m, 1H), 7.72-7.70 (m, 1H), 7.64-7.60 (m, 1H) |
| 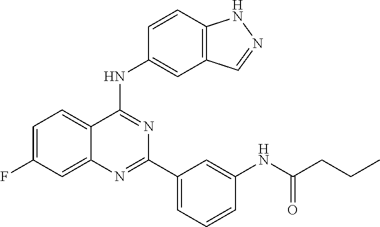 [SMILES: CCCC(=O)Nc1cccc(-c2nc(Nc3ccc4[nH]ncc4c3)c3ccc(F)cc3n2)c1] | 20 | [441.31 M]+ | 13.08 (m, 1H), 10.06 (m), 8.79 (s, 1H), 8.75-8.71 (m, 1H), 8.43 (s, 1H), 8.23 (s, 1H), 8.05-8.03 (d, 1H), 7.86- 7.84 (dd, 1H), 7.69-7.58 (m, 4 H), 7.44-7,41 (m, 1H), 2.37 (m, 2H), 1.71- 1.66 (m, 1H), 0.98-0.94 (t, 3H) |
| 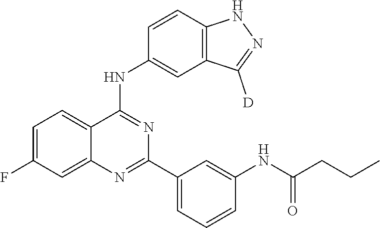 [SMILES: [2H]c1n[nH]c2ccc(Nc3nc(-c4cccc(NC(=O)CCC)c4)nc4cc(F)ccc34)cc12] | 21 | 442.22 [M]+ | 13-10-13.06 (m, 1H), 10.381 (s, 1H), 8.77 (m, 2H), 8.39 (m, 1H), 8.03-8.01 (d, 1H), 7.85-7.83 (m, 1H), 7.68-7.60 (m, 4H), 7.46-7.42 (m, 1H), 2.33-2.32 (m, 2H), 1.69-1.64 (m, 2H), 0.96-0.92 (t, 3H) |
| 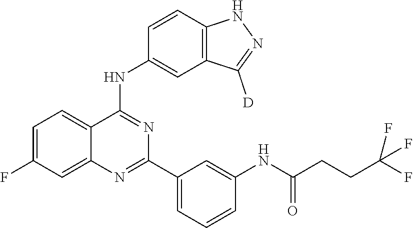 [SMILES: [2H]c1n[nH]c2ccc(Nc3nc(-c4cccc(NC(=O)CCC(F)(F)F)c4)nc4cc(F)ccc34)cc12] | 22 | 496.35 [M]+ | 10.42 (s, 1H), 8.93-8.89 (m, 1H), 8.74 (m, 1H), 8.33 (s, 1H), 8.05-8.03 (d, 1H), 7.85-7.82 (m, 2H), 7.72-7.68 (m, 3H), 7.55-7.51 (m, 1H), 2.67-2.62 (m, 4H) |
| 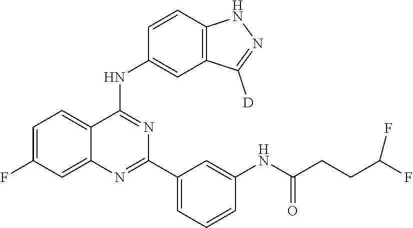 [SMILES: [2H]c1n[nH]c2ccc(Nc3nc(-c4cccc(NC(=O)CCC(F)F)c4)nc4cc(F)ccc34)cc12] | 23 | 478.33 [M]+ | 10.35 (s, 1H), 8.92-8.88 (m, 1H), 8.74 (s, 1H), 8.34 (s, 1H), 8.03-8.01 (m, 1H), 7.85-7.82 (m, 2H), 7.72-7.67 (m, 3H), 7.54-7.50 (m, 1H), 6.34-6.04 (m, 1H), 2.58-2.55 (m, 2H), 2.27-2.13 (m, 2H) |
| 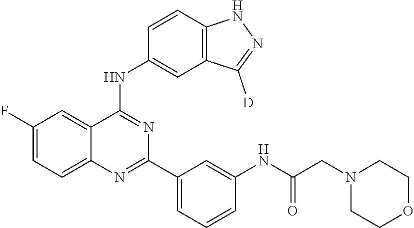 [SMILES: [2H]c1n[nH]c2ccc(Nc3nc(-c4cccc(NC(=O)CN5CCOCC5)c4)nc4ccc(F)cc34)cc12] | 24 | 499.30 [M]+ | 11.16 (m, 1H), 10.69 (m, 1H), 8.83- 8.81 (m, 1H), 8.71 (s, 1H), 8.32 (s, 1H), 8.24 (m, 1H), 8.18-8.16 (m, 1H), 7.98-7.95 (m, 1H), 7.87-7.84 (dd, 1H), 7.80-7.78 (m, 1H), 7.72-7.70 (m, 1H), 7.61-7.57 (m, 1H), 4.27 (s, 2H), 3.49 (m, 2H), 3.33 (m, 2H) |

### Example 2—Inhibition of ROCK-II Activity

Representative compounds of the invention were synthesized according to the described methods and tested for the ability to inhibit human Rho-associated protein kinase α (ROCK2) via a radiometric HotSpot™ kinase assay (see Anastassiadis T, et al. Comprehensive assay of kinase catalytic activity reveals features of kinase inhibitor selectivity. Nat Biotechnol. 2011 Oct. 30; 29(11):1039-45. doi: 10.1038/nbt.2017)
Assay Rection: Substrate+[γ-³³P]-ATP=³³P-Substrate+ADP
Assay Reaction Buffer: 20 mM Hepes (pH 7.5), 10 mM MgCl2, 1 mM EGTA, 0.01% Brij35, 0.02 mg/ml BSA, 0.1 mM Na3VO4, 2 mM DTT, 1% DMSO
Assay Procedure: Prepare peptide substrate

[KEAKEKRQEQIAKRRRLSSLRASTSKSGGSQK](20 μM) in freshly prepared Reaction Buffer. Deliver ROCK2 kinase into the substrate solution and gently mix. Deliver compounds in 100% DMSO into the kinase reaction mixture by Acoustic technology (Echo550; nanoliter range) and incubate for 20 min at room temp. Deliver ³³P-ATP into the reaction mixture to initiate the reaction and incubate for 2 hours at room temperature. Detect kinase activity by P81 filter-binding methodology.

Representative examples of the invention were tested for their ability to inhibit ROCK-2 mediated phosphorylation of peptide substrate. The inhibitory activity of representative examples is presented below. The Activity Range is: “++++” (IC₅₀<0.1 μM), “+++” (IC₅₀ 0.1 to 1.0 μM), “++” (IC₅₀ 1.0 to 10.0 μM) or “+” (IC₅₀>10.0 μM)

| Example No. | RST ID | ROCK2 Activity |
|---|---|---|
| 4 | 3014 | ++++ |
| 6 | 3008 | +++ |
| 16 | 3005 | +++ |
| 17 | 3010 | +++ |
| 18 | 3015 | +++ |
| 19 | 3020 | ++++ |
| 20 | 3007 | ++++ |
| 21 | 3013 | ++++ |
| 22 | 3011 | +++ |
| 23 | 3012 | +++ |
| 24 | 3019 | ++++ |

ROCK-II inhibitory activity can also be measured using the ROCK-II Assay Kit (Molecular Devices, inc.; Sunnyvale, Calif), for example:

### 2. A Functional Measure of ROCK Activity in Cells

### MLC Phosphorylation:

Myosin regulatory light chain phosphorylation can be measured in vascular smooth muscle (VSM) cells. VSM cells are isolated from the pulmonary artery of newborn calves and used in the 2nd to 4th passage. Cells are maintained in low glucose DME (JRH Biosciences) supplemented with 2 mM glutamine, 100 U/ml penicillin 100 U/ml streptomycin, 10 mM Hepes (Life Technologies), and 10% fetal bovine serum (Hyclone) in 10% CO2. Confluent monolayers are serum-starved for 72 hours in DME containing 0.4% fetal bovine serum prior to experiments. Quiescent cell monolayers are dissociated into single cells and plated at low. For experimental manipulation, cells are plated in DME containing 1% bovine serum albumin, transferrin (5 g/ml; Collaborative Research), human high density lipoprotein (10 g/ml; Intracel), 20 mM Hepes, sodium pyruvate (110 mg/L), penicillin G (100 units/ml), streptomycin (100 g/ml) and L-glutamine (0.292 mg/ml). Cells are harvested in ice-cold 10% trichloroacetic acid supplemented with 10 mM dithiothreitol (Sigma) and centrifuged at 13,000 rpm for 15 minutes at 4° C. The pellets are washed once with ice cold distilled water, and once with cold acetone. Samples are then placed in sample buffer (10 M urea [#161-0730, Bio-Rad], 1×Tris-glycine running buffer, 150 mM dithiothreitol, 0.01% bromophenol blue), sonicated, loaded onto and run on electrophoretic gels at 6 mA. Proteins are transferred to nitrocellulose in 1×Tris/glycine buffer with 20% methanol, blocked in three percent bovine serum albumin in Tris Buffered Saline, and probed with antibodies to detect phosphorylated isoforms of myosin regulatory light chain (Cell Signaling Technologies) for two hours at room temperature. Signals are detected using a horseradish peroxidase-conjugated secondary antibody (NA-131, Amersham; 1:4000) and Renaissance Enhanced Luminol Reagent (NEN Life Sciences Products) as a chemiluminescent substrate. Signal intensity is normalized and analyzed using NIH Image.

Cellular motility can be assessed using a migration assay. Fluorescently-labeled HT1080 human fibrosarcoma cells are seeded into a Fluoroblok Transwell 8 μM pore 96-well plate (Becton Dickenson) at a density of 40,000 cells per well in serum-free, phenol-free MEM. Compounds are added to the cells in the transwell inserts at a final concentration of 0.5% dimethylsulfoxide. Compounds are also added to the bottom wells in phenol-free MEM containing 10% fetal bovine serum as the chemoattractant. Cells are incubated at 37° C. for 4 h, and fluorescence is measured from the bottom of the plate on a fluorescent plate reader (Analyst, LJL Biosystems).

### Neovascularization in HUVEC Cells: In Vitro Endothelial Tube Formation Assay

### Materials

Matrigel® Matrix, 10 mL (Corning Cat. No. 354234)
24 well flat-bottom standard tissue culture-treated plate
Endothelial cell culture medium
Fetal bovine serum (FBS)
Vascular endothelial growth factor (VEGF)
Calcein AM Fluorescent Dye
Primary human endothelial cells (HUVEC)

### Procedure

Coat 24-well culture plate with Matrigel®. Prepare suspension of HUVECs in endothelial cell culture medium containing 10% FBS and VEGF (20 ng/mL). Add the cells (1×10⁵/well) to each well coated with Matrigel® and add test compounds. Incubate overnight at 37° C., 5% CO₂. Measure tube formation by first labelling the cells with calcein AM fluorescent dye and imaging using a fluorescent microscope.

### Example 3—Inhibition of CK2

Base Reaction buffer: 20 mM Hepes (pH 7.5), 10 mM MgCl2, 1 mM EGTA, 0.01%, Brij35, 0.02 mg/ml BSA, 0.1 mM Na3VO4, 2 mM DTT, 1% DMSO
Substrate: Peptide [RRRDDDSDDD]
ProQinase, catalog #0124-0000-1

### Reaction Procedure:

1. Prepare the peptide substrate stock in freshly prepared Reaction Buffer
2. Add any required cofactors to the substrate solution to achieve ATP to 10 μM and substrate concentration of 20 μM
3. Add full-length human CK2a (ProQinase, catalog #0124-0000-1) into the substrate solution and gently mix
4. Add test compounds into the kinase reaction mixture by Acoustic technology (Echo550; nanoliter range), incubate for 20 min at room temp
5. Deliver 33P-ATP into the reaction mixture to initiate the reaction
6. Incubate for 2 hours at room temperature
7. Detect kinase activity by P81 filter-binding method

Representative compounds of the invention tested under conditions described herein demonstrated good to excellent inhibition of CK2 activity (data not shown).

### Example 4—AMPK Activity by Western Blot

### Materials:

HT-1080 cell line was purchased from American Type Culture Collection (Manassas, VA). The cells were grown in EMEM medium, which was supplemented with 10% FBS, 100 μg/ml penicillin, and 100 μg/ml streptomycin. Culture was maintained at 37° C. in a humidified atmosphere of 5% CO2 and 95% air. 4-12% Bis-Tris gel and nitrocellulose membranes were purchased from Thermo Fisher Scientific (Waltham, MA). The Phospho-AMPKα (Thr172) (40H9) antibody (Cat #2535S), AMPKα (Cat #2532S) antibody, Protease/Phosphatase Inhibitor Cocktail (CST #5872S) and Cell Lysis Buffer (10×) (CST #9803) were purchased from Cell Signaling Technology (Danvers, MA). Anti-α-tubulin (T9026) antibody was purchased from Sigma (St. Louis, MO). Anti-rabbit IgG TRDye 680RD and anti-mouse IgG IRDye 800CW secondary antibodies were purchased from LI-COR (Lincoln, NE).

### Procedure:

1. 5×105 cells/well of HT-1080 cells were seeded in 12-well plates with complete culture medium overnight.
2. Next day the cells were treated with test compounds. 1 mM of positive control compound AICAR for 4 hours. DMSO was used as vehicle control.
3. The culture medium was removed, and the cells were washed with ice cold PBS.
4. The cells were lysed with 1× Cell Signaling Lysis Buffer supplemented with 1× proteinase/Phosphatase inhibitor.
5. Samples were sonicated briefly on ice.
6. 4× LDS sample buffer containing 200 mM DTT was added to the samples.
7. 35 ul of cell lysate per sample was separated by SDS-PAGE on 4-12% Bis-Tris gel and transferred onto nitrocellulose membrane by iBlot Dry Blotting system (Thermo Fisher Scientific).
8. The membrane was blocked with 1% milk for 1 hour, then probed with Phospho-AMPKα (Thr172) (40H9) antibody or AMPKα antibody in 1% milk overnight and re-probed with anti-α-tubulin antibody in 1% milk.
9. LI-COR anti-rabbit IgG IRDye 680RD and anti-mouse IgG IRDye 800CW secondary antibodies were used to detect the primary antibodies.
10. The membranes were scanned with LI-COR Odyssey Fc Imaging System. The specific bands of interest were quantified by LI-COR Image Studio Lite software.
11. Bar graphs were plotted using the GraphPad Prism 4 program.

Representative compounds of the invention tested under conditions described herein demonstrated good to excellent AMPK properties (data not shown).

### INCORPORATION BY REFERENCE

All of the patents and publications cited herein are hereby incorporated by reference in their entireties.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I): 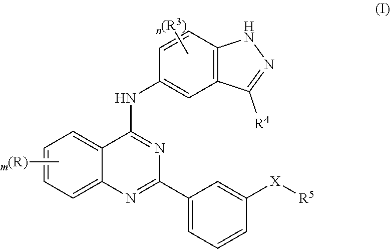
or a pharmaceutically acceptable salt thereof, wherein:
each R is independently H, deuterium, halogen, C₁₋₆ alkyl, C₁₋₆ haloalkyl, C₃₋₆ carbocyclyl, 3-6 membered heterocyclyl, or —OR⁷, wherein each alkyl, haloalkyl, carbocyclyl, or heterocyclyl is independently optionally substituted, wherein at least one R is selected from deuterium, halogen, alkoxy, trifluoromethyloxy or nitrile;
each R³ is independently deuterium, halogen, C₁₋₆ alkyl, C₁₋₆ haloalkyl, C₃₋₆ carbocyclyl, 3-6 membered heterocyclyl, or —OR⁷, wherein each alkyl, haloalkyl, carbocyclyl, or heterocyclyl is independently optionally substituted;
n is 0, 1, 2, or 3;
m is 0, 1, 2, 3 or 4;
X is —CON(R⁶)— or —N(R⁶)CO—
R⁵ and R⁶ are each independently H, C₁₋₆ alkyl, C₁₋₆ haloalkyl, aryl, heteroaryl, C₃₋₆ carbocyclyl, or 3-6 membered heterocyclyl, or R⁵ and R⁶ are joined together to form 3-6 membered heterocyclyl, wherein each alkyl, haloalkyl, aryl, heteroaryl, carbocyclyl, or heterocyclyl is independently optionally substituted; and
each R⁷ is independently H, C₁₋₆ alkyl, C₁₋₆ haloalkyl, C₃₋₆ carbocyclyl, or 3-6 membered heterocyclyl, wherein each alkyl, haloalkyl, carbocyclyl, or heterocyclyl is independently optionally substituted.

2. The compound of claim 1, wherein the compound is of Formula II: 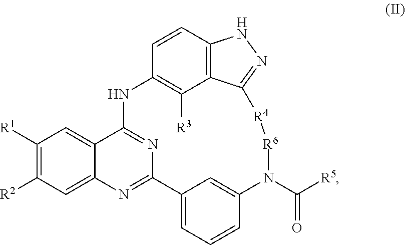
or a pharmaceutically acceptable salt thereof wherein R¹ and R² are independently selected from hydrogen, deuterium, halogen, methoxy, trifluoromethyloxy or nitrile.

3. The compound of claim 2, wherein R² is hydrogen.

4. The compound of claim 2, wherein R¹ is selected from halogen (fluorine, chlorine), methoxy, trifluoromethyloxy, and nitrile.

5. The compound of claim 1, wherein R³ is fluorine or hydrogen, preferably hydrogen.

6. The compound of claim 1, wherein R⁴ is deuterium or hydrogen, preferably hydrogen.

7. The compound of claim 1, wherein R⁶ is hydrogen and R⁵ is an optionally substituted alkyl.

8. The compound of claim 7, wherein R⁵ is C₁₋₆ alkyl optionally substituted by fluorine, such as propyl, 3, 3-difluoropropyl, 3,3,3-trifluoropropyl, 3, 3,-difluoro, 1-methylpropyl, cyclopropylmethyl.

9. The compound of claim 1, or a pharmaceutically acceptable salt thereof, wherein the compound has the Formula III: 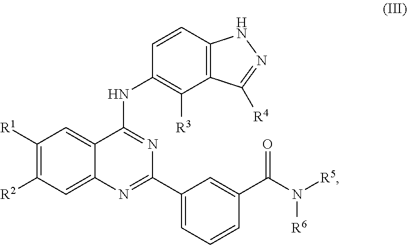
or a pharmaceutically acceptable salt thereof wherein R¹ and R² are independently selected from hydrogen, deuterium, halogen, methoxy or trifluoromethyloxy.

10. The compound of claim 9, wherein R² is hydrogen.

11. The compound of claim 9, wherein R¹ is selected from is selected from halogen (fluorine, chlorine), methoxy, trifluoromethyloxy, and nitrile.

12. The compound of claim 9, wherein R³ is fluorine or hydrogen, preferably hydrogen.

13. The compound of claim 9, wherein R⁴ is deuterium or hydrogen, preferably hydrogen.

14. The compound of claim 9, wherein R⁶ is hydrogen and R⁵ is an optionally substituted alkyl.

15. The compound of claim 9, wherein R⁵ is C₁₋₆ alkyl optionally substituted by fluorine, such as propyl, 3, 3-difluoropropyl, 3,3,3-trifluoropropyl, 3, 3,-difluoro, 1-methylpropyl, cyclopropylmethyl.

16. The compound of claim 9, wherein R⁵ is C₁₋₆ alkyl substituted by a —CONR⁸R⁹ group wherein R⁸ and R⁹ are each independently H, C₁₋₆ alkyl, C₁₋₆ haloalkyl, C₃₋₆ carbocyclyl, or 3-6 membered heterocyclyl, or, preferably, R⁸ and R⁹ are joined together to form an optionally substituted 3-6 membered heterocyclyl, such as

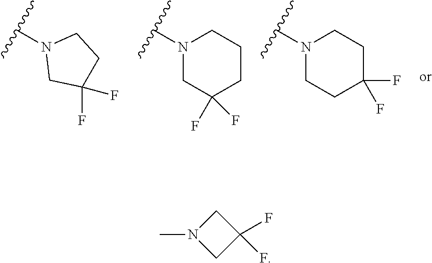

17. The compound of claim 9, wherein R⁶ is hydrogen and R⁵ is an optionally substituted aryl or heteroaryl, such as phenyl.

18. The compound of claim 17, wherein R⁵ is a phenyl substituted by fluorine.

19. The compound of claim 17, wherein R⁵ is a phenyl substituted by a —CONR⁸R⁹ group wherein R⁸ and R⁹ are each independently H, C₁₋₆ alkyl, C₁₋₆ haloalkyl, C₃₋₆ carbocyclyl, or 3-6 membered heterocyclyl, or, preferably, R⁸ and R⁹ are joined together to form an optionally substituted 3-6 membered heterocyclyl, such as:

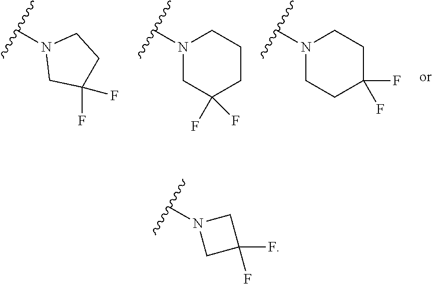

20. The compound of claim 9, wherein R⁵ and R⁶ are taken together to form an optionally substituted 5 membered heteroaryl.

21. The compound of claim 20, wherein R⁵ and R⁶ are taken together to form an optionally substituted 5 membered heteroaryl substituted by a —CONR⁸R⁹ group wherein R⁸ and R⁹ are each independently H, C₁₋₆ alkyl, C₁₋₆ haloalkyl, C₃₋₆ carbocyclyl, or 3-6 membered heterocyclyl, or, preferably, R⁸ and R⁹ are joined together to form an optionally substituted 3-6 membered heterocyclyl,

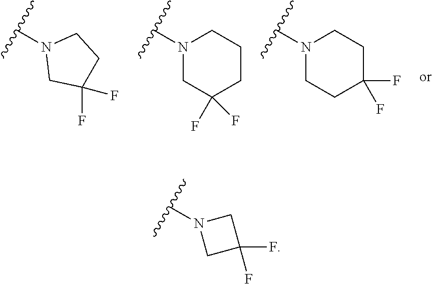

22. The compound of claim 1, wherein the compound or a pharmaceutically acceptable salt thereof is selected from the group consisting of:

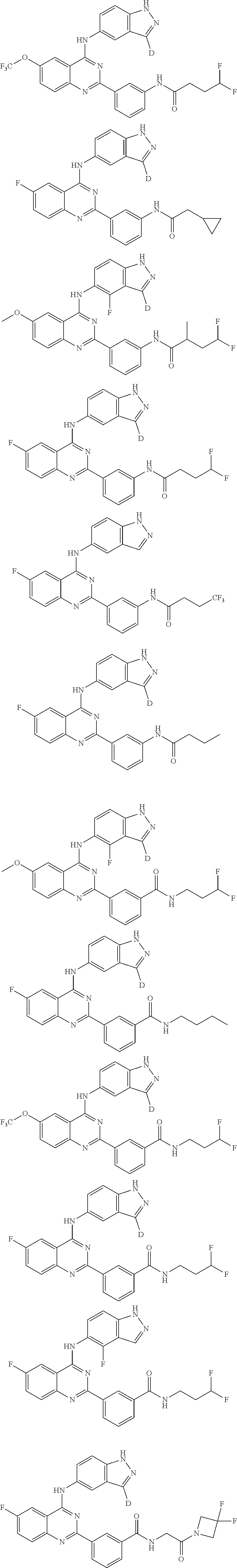

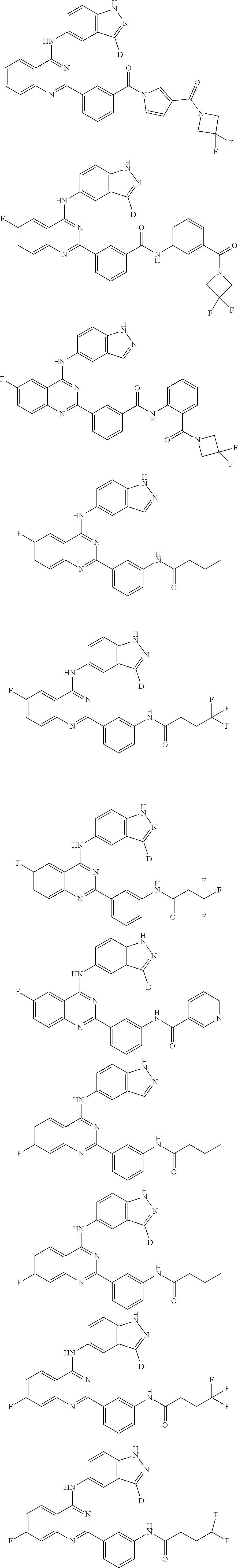

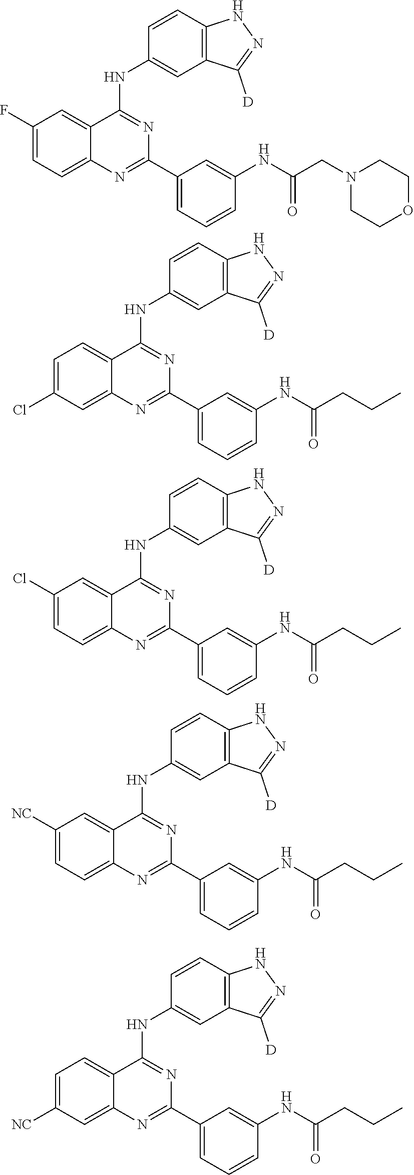

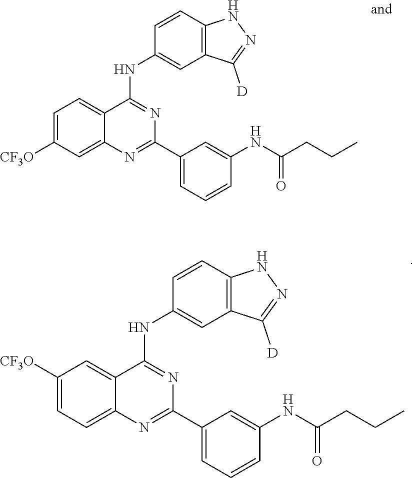

23. A pharmaceutical composition comprising a compound of claim 1, or a pharmaceutically acceptable salt thereof, and a pharmaceutically acceptable carrier or excipient.

24. A method of treating a disease associated with ROCK2 activity in a subject in need thereof comprising administering to the subject a therapeutic amount of a compound of claim 1, or a pharmaceutically acceptable salt thereof, or a pharmaceutical composition thereof.

25. A method of treating a disease associated with CK2 activity in a subject in need thereof comprising administering to the subject a therapeutic amount of a compound of claim 1, or a pharmaceutically acceptable salt thereof, or a pharmaceutical composition thereof.

26. A method of treating a disease in a subject in need thereof comprising administering to the subject a therapeutic amount of a compound of claim 1, or a pharmaceutically acceptable salt thereof, or a pharmaceutical composition thereof, wherein the disease is selected from fibrotic diseases, inflammatory diseases, autoimmune diseases, cardiovascular disorders, central nervous system disorders, neoplastic diseases, metabolic syndromes, ocular diseases, renal diseases, pulmonary diseases, muscular dystrophy, sickle cell disease, and viral diseases.

27-31. (canceled)

32. A method of inhibiting ROCK2 activity comprising contacting a ROCK2 protein with a compound of claim 1, or a pharmaceutically acceptable salt thereof, or a pharmaceutical composition thereof.

33. A method of inhibiting CK2 activity comprising contacting a CK2 protein with a compound of claim 1, or a pharmaceutically acceptable salt thereof, or a pharmaceutical composition thereof.

34-36. (canceled)
